SEQUENCE LISTING (1 sequence)

SEQ ID NO: 1 - protein, 17 aa, Pseudomonas aeruginosa
TVTRALRDLMQGILQKI

(19) United States
(12) Patent Application Publication
(10) Pub. No.: US 2022/0008376 A1
(43) Pub. Date: Jan. 13, 2022

(54) INHIBITORS OF TYPE 3 SECRETION SYSTEM AND ANTIBIOTIC THERAPY

(71) Applicant: University of Maryland, Baltimore, Baltimore, MD (US)

(72) Inventor: Wuyuan LU, Clarkville, MD (US)

(21) Appl. No.: 17/290,691

(22) PCT Filed: Nov. 1, 2019
(86) PCT No.: PCT/US19/59500

§ 371 Date: Apr. 30, 2021

Related U.S. Application Data
(60) Provisional application No. 62/754,960, filed on Nov. 2, 2018.

(51) Int. Cl. A61K 31/343; C12Q 1/18; A61P 31/04

(52) U.S. Cl. CPC A61K 31/343; A61P 31/04; C12Q 1/18

(57) ABSTRACT

Type 3 Secretion System (T3 SS) inhibitors, tanshinones and tanshinone analogs, and methods of using the same for the treatment of disease are disclosed. The invention relates generally to antibiotic compounds and methods of treating or preventing bacterial infections using the same, and more particularly, but not exclusively, to compounds that inhibit biogenesis of the Type 3 Secretion System (T3 SS) needle, including tanshinone and tanshinone analogs, and methods of using the same.

### CROSS-REFERENCE

This application claims benefit of priority to U.S. Provisional Application No. 62/754,960, filed on Nov. 2, 2018, which application is incorporated herein by reference in its entirety.

### STATEMENT AS TO FEDERALLY SPONSORED RESEARCH

This invention was made with government support under Grant Numbers GM106710 and CA219150 awarded by the National Institutes of Health. The government has certain rights in the invention.

### FIELD OF THE INVENTION

The invention relates generally to antibiotic compounds and methods of treating or preventing bacterial infections using the same, and more particularly, but not exclusively, to compounds that inhibit biogenesis of the Type 3 Secretion System (T3 SS) needle, including tanshinone and tanshinone analogs, and methods of using the same.

### BACKGROUND OF THE INVENTION

Antimicrobial resistance is becoming one of the greatest threats to public health. According to a widely cited authoritative report, drug resistant infections by bacteria, viruses and fungi will cause 10 million annual deaths worldwide by 2050, underscoring an urgent need to develop new classes of therapeutics to avert this global crisis. Bacteria develop drug resistance by controlling the uptake or efflux of antibiotics via altered membrane permeability, enzymatically inactivating them, or modifying their intended intervention targets, among other mechanisms. Conventional antibiotics aim to kill, thus subjecting bacteria to evolutionary selection pressure that invariably induces drug-resistant mutations to escape the killing. In some way, ideal antibiotics are refractory to existing resistance mechanisms and can block the ability of bacteria to infect hosts without directly killing them, thus avoiding inducing drug resistance. Toward this end, targeting bacterial virulence factors that are non-essential for survival but critical for pathogenicity has emerged as one of the most attractive strategies to combat antibiotic resistance. This anti-virulence strategy, in principle, ensues a low likelihood for bacteria to develop resistance as it induces less selective pressure on them. One such virulence factor of many pathogenic Gram-negative bacteria is the Type 3 Secretion System (T3SS).

### SUMMARY OF THE INVENTION

The disclosure provides in one aspect a method of treating or preventing a Gram-negative bacterial infection in a subject in need thereof, the method comprising administering to the subject a therapeutically effective amount of an inhibitor of Type 3 Secretion System (T3SS), wherein the inhibitor of T3SS is selected from the group consisting of a tanshinone, tanshinone analog, and the pharmaceutically acceptable salts, solvates, hydrates, cocrystals, or prodrugs thereof. In one embodiment, the tanshinone is tanshinone 1 (TSN1). In some embodiments, the tanshinone analog is dihydrotanshinone 1 (dHTSN1) or dihydrotanshinone (dHTSN).

In another aspect there is provided a pharmaceutical composition for the treatment or prevention of a Gram-negative bacterial infection in a subject in need thereof, the composition comprising an inhibitor of Type 3 Secretion System (T3SS), wherein the inhibitor of T3SS is selected from the group consisting of a tanshinone, tanshinone analog, and the pharmaceutically acceptable salts, solvates, hydrates, cocrystals, or prodrugs thereof.

In yet another aspect, there is provided a method of inhibiting treating or preventing a Gram-negative bacterial infection in a subject in need thereof, the method comprising administering to the subject a therapeutically effective amount of an inhibitor of Type 3 Secretion System (T3SS), wherein the inhibitor of T3SS blocks interaction between a T3SS needle protein and a T3SS chaperone protein.

In another aspect, the disclosure provides a pharmaceutical composition for the treatment or prevention of a Gram-negative bacterial infection in a subject in need thereof, the composition comprising an inhibitor of Type 3 Secretion System (T3SS), and a pharmaceutically acceptable carrier, wherein the inhibitor of T3SS blocks interaction between a T3SS needle protein and a T3SS chaperone protein.

In yet another aspect, there is provided a method of identifying an inhibitor of Type 3 Secretion System (T3SS), the method comprising:
(a) adding a candidate agent to a composition comprising a protein complex in the T3SS, wherein a component of the protein complex is fluorescently labeled;
(b) determining the fluorescence polarization (FP) of the fluorescently labeled component;
wherein the candidate agent is identified as an inhibitor of T3SS if the FP is decreased relative to a reference FP level.

In another aspect, the disclosure provides a method of treating or preventing a Gram-negative bacterial infection in a subject in need thereof, the method comprising administering to the subject a therapeutically effective amount of an agent identified as an inhibitor of Type 3 Secretion System (T3SS) according to the method of identifying T3SS inhibitors as described herein.

In another aspect, there is provided a pharmaceutical composition for the treatment or prevention of a Gram-negative bacterial infection in a subject in need thereof, the composition comprising an agent identified as an inhibitor of Type 3 Secretion System (T3SS) according to the methods of identifying T3SS inhibitors describe herein, and a pharmaceutically acceptable carrier.

In one embodiment of any one of the aspects herein, the Gram-negative bacteria is Pseudomonas, Escherichia, Salmonella, Shigella, Yersinia, Vibrio, Burkholderia, or Chlamydia. In yet another embodiment, the Gram-negative bacteria is Escherichia coli or Pseudomonas aeruginosa. In another embodiment, the bacterial infection is a lung infection, skin infection, soft tissue infection, gastrointestinal infection, urinary tract infection, meningitis, or sepsis. In one embodiment, the bacterial infection is caused by and/or associated with Pseudomonas aeruginosa. In one embodiment, the bacterial infection is pneumonia. In one embodiment, the subject is human.

In one aspect, there is provided a method of treating a Gram-negative bacterial infection in a subject in need thereof, the method comprising administering to the subject a therapeutically effective amount of an inhibitor of Type 3 Secretion System (T3SS) selected from the group consisting of tanshinone 1 (TSN1), dihydrotanshinone 1 (dHTSN1), dihydrotanshinone (dHTSN), and the pharmaceutically acceptable salts, solvates, hydrates, cocrystals, or prodrugs thereof.

In another aspect, there is provided a pharmaceutical composition for the treatment of a Gram-negative bacterial infection in a subject in need thereof, the composition comprising an inhibitor of Type 3 Secretion System (T3SS) selected from the group consisting of tanshinone 1 (TSN1), dihydrotanshinone 1 (dHTSN1), dihydrotanshinone (dHTSN), and the pharmaceutically acceptable salts, solvates, hydrates, cocrystals, or prodrugs thereof; and a pharmaceutically acceptable carrier.

### BRIEF DESCRIPTION OF THE FIGURES

FIGS. 1A-1B illustrate biogenesis of the Pseudomonas aeruginosa T3SS needle. FIG. 1A shows a schematic representation of the T3SS of Pseudomonas aeruginosa, adapted from Abrusci et al. 2014. FIG. 1B shows a crystal structure of the heterotrimeric complex of PscE-PscF-PscG determined by Quinard et al. Proc Natl Acad Sci USA 2007, 104 (19), 7803-7808. Shown in red are residues 54-85 of PscF, which makes direct interactions with PscG (but not PscE). The major α-helix at the C-terminus of PscF energetically dictates PscF binding to the stable heterdimeric complex of PscE-PscG.

FIGS. 2A-2B illustrate characterization of synthetic peptides/proteins by HPLC, ESI-MS and CD spectroscopy. FIG. 2A shows chemically synthesized PscF⁵⁴⁻⁸⁵, PscE and PscG characterized by RP-HPLC and ESI-MS. RP-HPLC analyses were performed at 40° C. on a Waters XBridge C18 column (4.6×150 mm, 3.5 m) running a 30-min, 5-65% linear gradient of acetonitrile in water containing 0.1% TFA at a flow rate of 1 ml/min. The molecular masses were ascertained by ESI-MS, in agreement with the calculated values. FIG. 2B shows circular dichroism spectra obtained at 25° C. of synthetic PscE, PscF, PscG, PscE-PscG heterodimer and PscE-PscF-PscG heterotrimer at 20 μM each in 10 mM phosphate buffer, pH 7.4.

FIGS. 3A-3E illustrate identification of tanshinone derivatives as inhibitors of the biogenesis of the Pseudomonas aeruginosa T3SS needle. FIG. 3A shows a strategy for the design of a fluorescence polarization assay for high-throughput screening (HTS). The difference in fluorescence polarization between PscE-^{FAM}PscF-PscG (high) and ^{FAM}PscF (low) forms the basis of a physical readout for HTS. FIG. 3B shows representative quantification by isothermal titration calorimetry of the interaction of PscF⁶⁹⁻⁸⁵ with a preformed PscE-PscG heterodimer at 25° C. in 10 mM Tris, 150 mM NaCl, 1 mM EDTA, pH 7.0. FIG. 3C shows representative quantification by fluorescence polarization of the interaction of ^{FAM}PscF⁶⁹⁻⁸⁵ with a preformed PscE-PscG heterodimer at room temperature in 10 mM Tris, 150 mM NaCl, 1 mM EDTA, pH 7.0. FIG. 3D shows representative competition of PscF⁶⁹⁻⁸⁵, tanshinone 1 (TSN1), dihydrotanshinone 1 (dHTSN1) and dihydrotanshinone (dHTSN) with ^{FAM}PscF⁶⁹⁻⁸⁵ for binding to PscE-PscG heterodimer as quantified by fluorescence polarization at room temperature in 10 mM Tris, 150 mM NaCl, 1 mM EDTA, 1% DMSO, pH 7.0. FIG. 3E shows chemical structures of TSN1, dHTSN1 and dHTSN.

FIGS. 4A-4C show structural characterization of tanshinone derivatives interacting with PscE-PscG by NMR spectroscopy and molecular modeling. FIG. 4A shows the ¹⁵N-¹H HSQC spectra of ¹⁵N-labelled PscG of the PscE-PscG heterodimer in the presence (gray) and absence (black) of dHTSN1. Circled are the resonance peaks broadening or shifting upon binding to dHTSN1. Inset: the amide resonance peaks of tryptophan side-chains in ¹⁵N-labelled PscG. FIG. 4B shows the crystal structure of the PscE-PscF-PscG heterotrimer^{[15]} displaying four Trp residues of PscG (light gray), three of which, W67, W73 and W79, are located in the same α-helix involved in direct interactions with PscF (dark gray). FIG. 4C shows TSN1, dHTSN1 and dHTSN docked in the PscF-binding pocket of PscG. Molecular modeling identifies W79 as the most probable Trp residue involved in direct interactions with tanshinones.

FIGS. 5A-5B show functional characterization of tanshinone derivatives as inhibitors of the biogenesis of the Pseudomonas aeruginosa T3SS needle In vitro. FIG. 5A shows effects of tanshinone 1 (TSN1), dihydrotanshinone 1 (dHTSN1), dihydrotanshinone (dHTSN) and cryptotanshinone (crpTSN) at 100 μM on cell viability of Pseudomonas aeruginosa strain PAO1 and murine macrophage cell line J774A.1. The data are averages of three independent experiments. FIG. 5B shows effects of tanshinone compounds at 100 μM on the secretion of ExoS by PAO1 grown under low-calcium conditions where the T3SS is transcriptionally activated. The data are averages of three independent experiments. Note that tanshinones were initially dissolved in DMSO and diluted into culture medium for in vitro assays, where 2% DMSO in culture medium was used as negative control.

FIGS. 6A-6C illustrate functional characterization of tanshinone derivatives as inhibitors of the biogenesis of the Pseudomonas aeruginosa T3SS needle in vitro. FIG. 6A shows inhibition of the cytotoxicity of PAO1 to murine macrophages by different concentrations of tanshinone compounds as measured by the LDH release assay. The data are averages of three independent experiments. FIG. 6B shows a Western blot analysis of caspase 1 activation in PAO1-infected murine macrophages treated with tanshionone compounds at 100 μM. FIG. 6C shows inhibition of intracellular proliferation of PAO1 in murine macrophages by different concentrations of tanshinone compounds. The data are averages of three independent experiments. Note that tanshinones were initially dissolved in DMSO and diluted into culture medium for in vitro assays, where 2% DMSO in culture medium was used as negative control.

FIGS. 7A-7C show functional characterization of tanshinone derivatives as inhibitors of the biogenesis of the Pseudomonas aeruginosa T3SS needle in vivo. FIG. 7A shows effects of TSN1, dHTSN and dHTSN 1 on the survival of C57BL/6J mice (n=20 in each group) intranasally challenged with PAO1. Left panel: tanshinones were administered simultaneously with PAO1; right panel: tanshinones were administered 8 h post infection. FIG. 7B shows reduction of bacterial burden in the bronchoalveolar lavage of PAO1-infected mice by tanshinone compounds. FIG. 7C shows H&E staining of the lungs from normal mice and PAO1-infected mice treated with tanshinone compounds and control. Note that tanshinones were initially dissolved in DMSO and diluted into PBS for in vivo assays, where 1% DMSO in PBS was used as negative control. The aqueous solubility of tanshinones in the presence of 1% DMSO ranges from 200 to 300 μM (FIG. 24).

FIG. 8 illustrates the strategy for the preparation of PscE, PscF and PscG using solid phase peptide synthesis coupled with native chemical ligation. To obtain the heterotrimeric complex, PscE, PscF and PscG at an equal molar ratio were dissolved in 6 M GuHCl followed by a 6-fold dilution with and an overnight dialysis against PBS.

FIGS. 9A-9C show PscF characterized by RP-HPLC and ESI-MS. FIG. 9A shows PscF⁵⁴⁻⁸⁵. FIG. 9B shows FAM-PscF⁶⁹⁻⁸⁵. FIG. 9C shows PscF⁶⁹⁻⁸⁵. RP-HPLC analyses were performed on a Waters XBridge C18 column (4.6×150 mm, 3.5 μm) running a 30-min gradient of acetonitrile from 5% to 65%. The molecular masses were ascertained by electrospray ionization mass spectrometry (ESI-MS).

FIGS. 10A-10C show PscE peptides characterized by RP-HPLC and ESI-MS. FIG. 10A shows PscE¹⁻⁴²αCOSR (R═CH₂CO-Leu-OH). FIG. 10B shows PscE⁴³⁻⁷⁰. FIG. 10C shows PscE¹⁻⁷⁰. RP-HPLC analyses were performed on a Waters XBridge C18 column (4.6×150 mm, 3.5 μm) running a 30-min gradient of acetonitrile from 5% to 65%. The molecular masses were ascertained by electrospray ionization mass spectrometry (ESI-MS).

FIGS. 11A-11C PscG peptides characterized by RP-HPLC and ESI-MS. FIG. 11A shows THZ-PscG²⁶⁻⁷⁵(CHO)₃αCOSR (R═CH₂CO-Leu-OH). FIG. 11B shows PscG⁷⁶⁻¹¹⁴CHO. FIG. 11C shows THZ-PscG²⁶⁻¹¹⁴(CHO)₄. RP-HPLC analyses were performed on a Waters XBridge C18 column (4.6×150 mm, 3.5 μm) running a 30-min gradient of acetonitrile from 5% to 65%. The molecular masses were ascertained by electrospray ionization mass spectrometry (ESI-MS).

FIGS. 12A-12D PscG peptides characterized by RP-HPLC and ESI-MS. FIG. 12A shows PscG²⁶⁻¹¹⁴(CHO)₄. FIG. 12B shows PscG¹⁻²⁵αCOSR (R═CH₂CO-Leu-OH). FIG. 12C shows PscG¹⁻¹¹⁴(CHO)₄. FIG. 12D shows PscG¹⁻¹¹⁴. RP-HPLC analyses were performed on a Waters XBridge C18 column (4.6×150 mm, 3.5 m) running a 30-min gradient of acetonitrile from 5% to 65%. The molecular masses were ascertained by electrospray ionization mass spectrometry (ESI-MS).

FIGS. 13A-13D illustrate characterization of PscE, PscF and PscG. FIG. 13A shows a MW Standard calibration curve obtained on the Superdex 75 column (10/300 GL) using conalbumin (75000), ovalbumin (43000), carbonic anhydrase (29000), ribonuclease A (13700) and aprotinin (6500). The buffer was 10 mM Tris, 150 mM NaCl, 1 mM EDTA, pH 7.0, running at a flow rate of 0.5 ml/ml at room temperature. FIG. 13B shows elution of PscE-PscF-PscG on Superdex 75. The apparent molecular weight calculated from the standard calibration curve indicates the presence of a heterotrimeric complex. FIG. 13C shows RP-HPLC of the heterotrimeric complex yields the three well-resolved peaks of PscE, PscF and PscG confirmed by ESI-MS. FIG. 13D is a plot showing thermal denaturation of PscG, PscE, PscF, PscG-PscE heterodimer and PscG-PscE-PscF heterotrimer. All peptides were prepared at 20 μM in PBS, monitored between 25° C. and 90° C. by CD spectroscopy at 222 nm. Nonlinear regression analyses yielded the melting temperature (Tm) values of 40.1±0.5 for PscF, 58.7±1.2 for PscE, 39.2±1.5 for PscG, 43.1±1.3 for PscE-PscG, 59.9±0.9 for PscF-PscG, and 61.7±0.7 for PscF-PscE-PscG.

FIGS. 14A-14C illustrate characterization of PscE, PscF and PscG. FIG. 14A shows quantification of the interaction between PscF⁶⁹⁻⁸⁵ and PscG by ITC in 10 mM Tris, 150 mM NaCl, 1 mM EDTA, pH 7.0. FIG. 14B shows quantification of the interaction between PscE and PscG by ITC in 10 mM Tris, 150 mM NaCl, 1 mM EDTA, pH 7.0. FIG. 14C shows quantification by fluorescence polarization of the interaction of FAM-PscF⁶⁹⁻⁸⁵ with PscG at room temperature in 10 mM Tris, 150 mM NaCl, 1 mM EDTA, pH 7.0.

FIGS. 15A-15D depict an initial FP screening of ten natural herbal compounds at four different concentrations: 1 μM (FIG. 15A), 10 μM (FIG. 15B), 100 μM (FIG. 15C), and 1 mM (FIG. 15D). Significance compared with NC group (mock treated) was calculated using an unpaired t test, and p values are as follows: *p<0.05, **p<0.01, and ***p<0.001. Ammonium glycyrrhizinate, astragaloside A, baicalein, curculigoside, ginsenoside Rb1, ginsenoside Re, osthol, panaxadiol, quercetin, and tanshinone 1.

FIG. 16A shows the effects of different concentrations of tanshinone 1 on the circular dichroism spectrum of 20 μM PscG-PscE-PscF heterotrimer obtained at 25° C. in 10 mM phosphate buffer, pH 7.4. The helicity of the PscF-PscE-PscG complex progressively decreases as the concentration of tanshinone 1 increases. FIGS. 16B-16C show thermal denaturation of the PscF⁶⁹⁻⁸⁵-PscE-PscG complex at 20 μM in PBS in the absence (FIG. 16B) and presence (FIG. 16C) of 300 μM tanshinone 1, monitored between 25 and 90° C. by CD spectroscopy at 222 nm. Nonlinear regression analyses yielded the Tm values of 54.8 and 52.3, respectively.

FIG. 17 shows tanshinone analogues tested in this work. Note: —, concentration-dependent characteristics in the FP competition assay not observed; ND, not done. Significance compared with mock treated group was calculated by unpaired t test, and p values are as follows: ns, not significant (p>0.05), *p<0.05, **p<0.01, and ***p<0.001.

FIGS. 18A-18C are a set of plots showing fluorescence spectra scanned from 400 nm to 800 nm with an excitation wavelength of 470 nm in 10 mM Tris buffer, 150 mM NaCl, 1 mM EDTA, 5% DMSO, pH 7.0. FIG. 18A shows tanshinone compounds alone at 200 μM, and 5% DMSO in Tris buffer was used as a control. FIG. 18B shows PscE-PscG at 100 nM in the presence of tanshinone compounds at 200 μM. FIG. 18C shows FAM-PscF⁶⁹⁻⁸⁵ at 100 nM in the presence of tanshinone compounds at 200 μM.

FIG. 19 shows SDS-PAGE analysis of recombinant PscG expressed in E. coli. S: soluble fraction. IB: inclusion bodies.

FIG. 20 illustrates molecular docking of the three active tanshinone compounds into the hydrophobic groove formed between PscG (light gray) and PscE (dark gray).

FIG. 21 is a plot showing cytotoxicity to murine macrophages of PAO1 and PAO1 ΔpscC in the presence of tanshinone compounds at 100 μM as determined by a lactate dehydrogenase (LDH) release assay. Three independent experiments were performed.

FIG. 22 shows cytotoxicity to murine macrophages of PAO1 in the presence of tanshinone compounds at 100 μM as determined by a lactate dehydrogenase (LDH) release assay. Data were normalized against PAO1 (in the absence of tanshinones) as the positive control and a negative control, PAO1 ΔpscC. Average results of three independent experiments are shown as mean±SD.

FIG. 23 shows histopathologic scores to each group of lungs tissue section based on the degree of inflammation. Data are shown as mean±SD of three independent experiments (n=10 for each group). Significance compared with mock treated group was calculated using an unpaired t test, and p values are as follows: *p<0.05, **p<0.01, and ***p<0.001.

FIG. 24 is a set of plots showing quantification of the aqueous solubility of tanshinones in 1% DMSO by RP-HPLC. Chromatograms were obtained on a Waters XBridge C18 column (4.6×150 mm, 3.5 m) running a 45-min gradient of acetonitrile from 5% to 95%.

FIGS. 25A-25G show characterization of MBX1641. FIG. 25A shows MBX1641 characterized by RP-HPLC and ESI-MS. The RP-HPLC analysis was performed on a Waters XBridge C4 column (4.6×150 mm, 3.5 μm) running a 30-min gradient of acetonitrile from 5% to 65%. The molecular mass was ascertained by electrospray ionization mass spectrometry (ESI-MS). FIG. 25B shows effects of MBX1641 on the cell viability of Pseudomonas aeruginosa strain PAO1 and murine macrophage cell line J774A.1. The data are averages of three independent experiments. FIG. 25C shows effects of MBX1641 at 6.25 μM on the secretion of ExoS by PAO1 grown under low-calcium conditions where the T3SS is transcriptionally activated. The data are averages of three independent experiments. FIG. 25D shows inhibition of the cytotoxicity of PAO1 to murine macrophages by MBX1641 as measured by the LDH release assay. The data are averages of three independent experiments. FIG. 25E shows inhibition of intracellular proliferation of PAO1 in murine macrophages by MBX1641. The data are averages of three independent experiments. FIG. 25F shows reduction of bacterial burden in the bronchoalveolar lavage of PAO1-infected mice by 100 μM MBX1641. FIG. 25G shows representative competition of PscF⁶⁹⁻⁸⁵ (black) or MBX1641 (gray) with FAM-PscF⁶⁹⁻⁸⁵ for binding to PscG-PscE heterodimer as quantified by fluorescence polarization at room temperature in 10 mM Tris, 150 mM NaCl, 1 mM EDTA, pH 7.0.

FIGS. 26A-26B show structures of exemplary tanshinone or tanshinone analog compounds.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include the compounds described herein and, more specifically: a T3SS inhibitor (e.g., a tanshinone or tanshinone analog, such as tanshinone 1 (TSN1), dihydrotanshinone 1 (dHTSN1) or dihydrotanshinone (dHTSN)). The terms “active pharmaceutical ingredient” and “drug” may also include those compounds described herein that bind a T3SS component, e.g., compounds that bind a T3SS needle protein or chaperone protein and block interaction between a T3SS chaperone and needle protein.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., increased sensitivity to apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. In some embodiments, the biological molecules modulated by the methods and compounds of the invention to effect treatment may include molecules in the Type 3 secretion system (T3SS), such as a T3SS needle protein (e.g., PscF) or a T3SS chaperone protein (e.g., PscE, PscG).

“Inhibitors” are agents that inhibit a recited activity, function or entity. A “T3SS inhibitor” or “inhibitor of T3SS” is an agent which reduces the function or activity of a Type 3 secretion system (T3SS). In some embodiments, the T3SS inhibitor prevents or reduces T3SS-induced bacterial virulence in vitro. In some embodiments, the T3SS inhibitor prevents or reduces T3SS-induced bacterial virulence in vivo. A T3SS inhibitor can be a compound which binds to a T3SS component and interfere with the binding of the T3SS component to other T3SS components (e.g., binding to the same T3SS protein to form homomultimers, or binding to a different T3SS protein to form heteromultimers). For example, the compound can bind to a T3SS protein and interfere with its multimerization. In some embodiments, the T3SS inhibitor competes for binding to a T3SS chaperone protein with a T3SS needle protein. In some embodiments, the T3SS inhibitors are provided in a composition also comprising a sterile carrier and/or physiologically acceptable carrier.

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of a tanshinone compound (e.g., tanshinone 1 (TSN1)) or tanshinone analog (e.g., dihydrotanshinone 1 (dHTSN1) or dihydrotanshinone (dHTSN)). The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the invention with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a tanshinone compound such as tanshinone 1 (TSN1), or tanshinone analog such as dihydrotanshinone 1 (dHTSN1) or dihydrotanshinone (dHTSN))) is a prodrug within the scope of the invention. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action, to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the invention.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

### Inhibitors of Type 3 Secretion System (T3SS)

The T3SS, found as cell-surface appendages, comprises ˜30 bacterial proteins⁷⁻⁹, which are classified into structural, effector and chaperon proteins whose respective functions are largely conserved across different bacterial species. The structural proteins of the T3SS polymerize into a membrane-anchored, needle-like assembly known as “the needle complex,” through which the effector proteins are injected from the bacterial cytoplasm into host cells to promote infection. Since the structural proteins are often hydrophobic and prone to aggregation on their own, they are bound and protected, prior to their high-order assembly on the bacterial membrane, by the chaperone proteins in the cytosol to prevent premature aggregation and degradation.

Pseudomonas aeruginosa is a resistance-prone, Gram-negative pathogen often found in the intensive care unit of a hospital. It causes life-threatening nosocomial infections such as pneumonia in immune-compromised patients, and poses a major risk of pulmonary deterioration to patients with chronic cystic fibrosis¹⁰. Various studies have demonstrated that virulence factors secreted via the T3 SS promote pathogenicity of Pseudomonas aeruginosa in vitro and in vivo, which correlates to poor clinical outcomes in Pseudomonas-infected patients.

In an embodiment, the invention includes compounds that are inhibitors of Type 3 Secretion System (T3SS). In an embodiment, the invention includes compounds that are inhibitors of the biogenesis or assembly of the T3SS needle.

As described herein, tanshinones were identified as first-in-class Inhibitors of the Biogenesis of the Type 3 Secretion System Needle of Pseudomonas aeruginosa for antibiotic therapy. The Type 3 Secretion System (T3SS) found as cell-surface appendages of many pathogenic Gram-negative bacteria, although non-essential for bacterial survival, is an important therapeutic target for drug discovery and development aimed at inhibiting bacterial virulence without inducing antibiotic resistance. Described herein is a design of a fluorescence polarization-based assay for high-throughput screening as a mechanistically well-defined general strategy for antibiotic discovery targeting the T3SS, and a serendipitous discovery of a subset of tanshinones—natural herbal compounds in traditional Chinese medicine widely used for the treatment of cardiovascular and cerebrovascular diseases—as effective inhibitors of the biogenesis of the T3 SS needle of multidrug-resistant Pseudomonas aeruginosa. By inhibiting T3SS needle assembly and, thus, cytotoxicity and pathogenicity, selected tanshinones reduced the secretion of bacterial virulence factors toxic to macrophages in vitro, and rescued experimental animals challenged with lethal doses of Pseudomonas aeruginosa in a murine model of acute pneumonia. As first-in-class inhibitors with a demonstrable safety profile in humans, tanshinones may be used directly to alleviate Pseudomonas aeruginosa-associated pulmonary infections without inducing antibiotic resistance. Since the T3SS is highly conserved among Gram-negative bacteria, this anti-virulence strategy may be applicable to the discovery and development of novel classes of antibiotics refractory to existing resistance mechanisms for the treatment of many bacterial infections.

In some embodiments, the compounds described herein may inhibit T3SS needle assembly. In some embodiments, the compounds described herein may reduce secretion of bacterial virulence factors. In some other embodiments, the compounds described herein reduce cytotoxicitiy and/or pathogencity of a bacteria. In some embodiments, the compounds described herein inhibit biogenesis of a T3SS needle and reduce secretion of bacterial virulence factors. In some embodiments, the compounds described herein may be delivered as a listed or as a pharmaceutically acceptable salt, solvate, hydrate, cocrystal, tautomer, or prodrug thereof

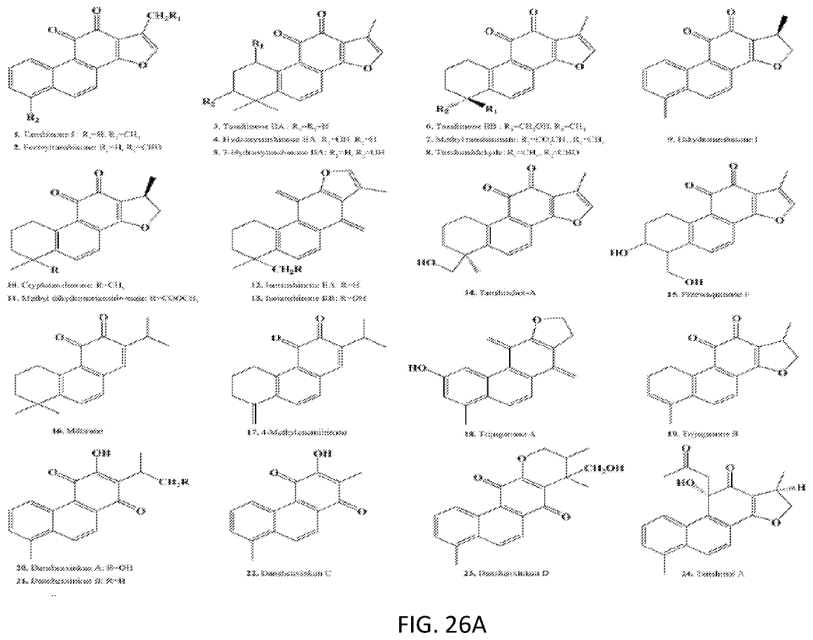

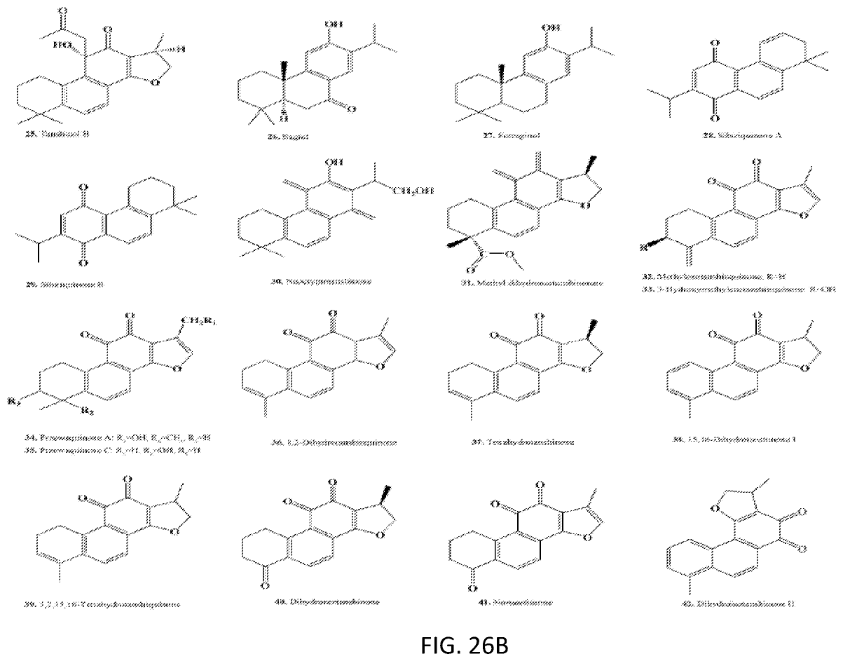

The disclosure provides tanshinone and tanshinone analog compounds. Tanshinones are a class of lipophilic phenanthrene compounds that are rich in the roots of S. miltiorrhiza. (Jiang et al., 2019, Frontiers in Pharmacology, Vol. 10, Article 202, pp. 1-14). The compounds possess an ortho- or fluorene structure and can be reduced to a diphenol derivative, which is converted to hydrazine after oxidation. Examples of tanshinones and tanshinone analogs include, without limitation, tanshinone I, formyltanshinone, tanshinone IIA, hydroxytanshinone IIA, 3-hydroxytanshinone IIA, tanshinone IIB, methyl tanshinonate, tanshinaldehyde, dihydrotanshinone I, cryptotanshinone, methyl dihydronortanshinonate, isotanshinone IIA, isotanshinone IIB, tanshindiol-A, przewaquinone F, miltirone, 4-methylenemiltirone, trijuganone A, trijuganone B, danshenxinkun A, danshenxinkun B, denshenxinkun C, denshenxinkun D, tanshinol A, tanshinol B, sugiol, ferruginol, sibiriquinone A, sibiriquinone B, neocryptotanshinone, methyl dihydronortanshinonate, methylenetanshinquinone, 3-hydroxymethylenetanshinquinone, przewaquinone A, przewaquinone C, 1,2-dihydrotanshinquinone, tetrahydrotanshinone, 15,16-dihydrotanshinone I, 1,2,15,16-tetrahydrotanshiquinone, dihydronortanshinone, nortanshionone, and dihydroisotanshinone II (FIGS. 26A-26B).

In some embodiments, the tanshinone or tanshinone analog is tanshinone I:

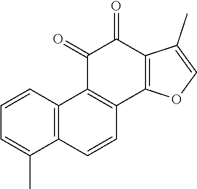 [SMILES: Cc1coc2c1C(=O)C(=O)c1c-2ccc2c(C)cccc12]

In some embodiments, the tanshinone or tanshinone analog is dihydrotanshinone 1 (dHTSN1):

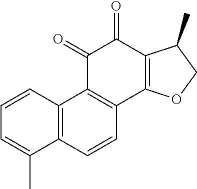 [SMILES: Cc1cccc2c3c(ccc12)C1=C(C(=O)C3=O)[C@@H](C)CO1]

In some embodiments, the tanshinone or tanshinone analog dihydrotanshinone (dHTSN):

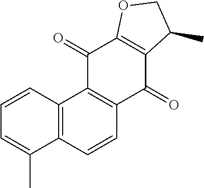 [SMILES: Cc1cccc2c3c(ccc12)C(=O)C1=C(OC[C@H]1C)C3=O]

### Methods of Treating Bacterial Infections and Other Diseases

The compounds and compositions described herein can be used in methods for treating diseases. In some embodiments, the compounds and compositions described herein can be used in methods for treating diseases associated with the Type 3 Secretion System (T3SS). In some embodiments, the compounds and compositions described herein can be used for the treatment of bacterial infections, including infections caused by and/or associated with Gram-negative bacteria. The compounds and compositions described herein may also be used in treating disorders as described herein and in the following paragraphs.

In one aspect, a method of treating or preventing a bacterial infection in a subject in need thereof is provided. In one aspect, a method of reducing virulence of a bacteria in a subject is provided. In another aspect, a method of reducing pathogenicity and/or cytoxicity of a bacteria in a subject is provided. In one aspect, a method of reducing or preventing development of drug resistance in a bacteria is provided. In some embodiments, the subject is an animal. In some embodiments, the subject is a human.

In one aspect a method of treating or preventing a bacterial infection in a subject in need thereof, is provided, the method comprising administering to the subject a therapeutically effective amount of an inhibitor of Type 3 Secretion System (T3SS). In another aspect, a method of treating or preventing a bacterial infection in a subject in need thereof, is provided, the method comprising administering to the subject a therapeutically effective amount of an agent identified as an inhibitor of Type 3 Secretion System (T3SS) according to the methods of identifying T3SS inhibitors as described herein. In another aspect, a method of inhibiting treating or preventing a bacterial infection in a subject in need thereof, is provided, the method comprising administering to the subject a therapeutically effective amount of an inhibitor of Type 3 Secretion System (T3SS), wherein the inhibitor of T3SS blocks interaction between a T3SS needle protein and a T3SS chaperone protein. In one embodiment, the bacterial infection is Gram-negative bacterial infection.

In one embodiment, the inhibitor of T3SS is selected from the group consisting of a tanshinone, tanshinone analog, and the pharmaceutically acceptable salts, solvates, hydrates, cocrystals, or prodrugs thereof. In one embodiment, the tanshinone is tanshinone 1 (TSN1). In some embodiments, the tanshinone analog is dihydrotanshinone 1 (dHTSN1) or dihydrotanshinone (dHTSN).

In an exemplary embodiment, an infection is caused by and/or associated with a bacteria. In an exemplary embodiment, the bacterium is a gram-positive bacteria. In another exemplary embodiment, the gram-positive bacterium is selected from the group consisting of Staphylococcus species, Streptococcus species, Bacillus species, Mycobacterium species, Corynebacterium species (Propionibacterium species), Clostridium species, Actinomyces species, Enterococcus species and Streptomyces species. In another exemplary embodiment, the gram-positive bacterium is selected from the group consisting of Propionibacterium acnes, Staphylococcus aureus, Staphylococcus epidermidis, Staphylococcus saprophyticus, Staphylococcus haemolyticus, Streptococcus pyogenes, Streptococcus agalactiae, Streptococcus pneumoniae, Enterococcus faecalis, Enterococcus faecium, Actinomyces israelii, Bacillus anthracis, Corynebacterium diphtheria, Clostridium perfringens, Clostridium botulinum, Clostridium tetani, and Clostridium difficile. In another exemplary embodiment, the gram-positive bacterium is selected from the group consisting of Staphylococcus aureus, Staphylococcus epidermidis, Streptococcus pneumoniae, Streptococcus pyogenes, Enterococcus faecalis, Enterococcus faecium, Clostridium difficile and Propionibacter acnes. In another exemplary embodiment, the bacterium is a gram-negative bacterium. In another exemplary embodiment, the gram-negative bacterium is selected from the group consisting of Acinetobacter species, Neisseria species, Pseudomonas species, Brucella species, Agrobacterium species, Bordetella species, Escherichia species, Shigella species, Yersinia species, Salmonella species, Klebsiella species, Enterobacter species, Haemophilus species, Pasteurella species, Streptobacillus species, spirochetal species, Campylobacter species, Vibrio species, Helicobacter species, Bacteroides species, Citrobacter species, Proteus species, Providencia species, Serratia species, Stenotrophomonas species and Burkholderia species. In another exemplary embodiment, the gram-negative bacterium is selected from the group consisting of Acinetobacter species, Pseudomonas species, Escherichia species, Klebsiella species, Enterobacter species, Bacteroides species, Citrobacter species, Proteus species, Providencia species, Serratia species, Stenotrophomonas species and Burkholderia species. In another exemplary embodiment, the gram-negative bacterium is selected from the group consisting of Neisseria gonorrhoeae, Neisseria meningitidis, Pseudomonas aeruginosa, Legionella pneumophila, Escherichia coli, Yersinia pestis, Haemophilus influenzae, Helicobacter pylori, Campylobacter fetus, Campylobacter jejuni, Vibrio cholerae, Vibrio parahemolyticus, Treponema pallidum, Rickettsia prowazekii, Rickettsia rickettsii, Chlamydia trachomatis, Chlamydia psittaci, Brucella abortus, Agrobacterium tumefaciens, Francisella tularensis, Klebsiella pneumoniae, Enterobacter cloacae, Acinetobacter baumannii, Bacteroides fragilis, Citrobacter freundii, Proteus mirabilis, Providencia stuartii, Serratia marcescens, Stenotrophomonas maltophilia and Burkholderia cepacia. In another exemplary embodiment, the gram-negative bacterium is selected from the group consisting of Pseudomonas aeruginosa, Escherichia coli, Haemophilus influenzae, Klebsiella pneumoniae, Enterobacter cloacae, Acinetobacter baumannii, Bacteroides fragilis, Citrobacter freundii, Proteus mirabilis, Providencia stuartii, Serratia marcescens, Stenotrophomonas maltophilia and Burkholderia cepacia. In another exemplary embodiment, the gram-negative bacterium is selected from the group consisting of Enterobacter aerogenes, Enterobacter cloacae, Enterobacter sakazakii, Escherichia coli, Klebsiella pneumoniae, Proteus mirabilis, Serratia marcescens and Citrobacter freundii. In another exemplary embodiment, the gram-negative bacterium is a Providencia spp.

In an exemplary embodiment, the bacteria is an acid-fast bacteria. In another exemplary embodiment, the bacterium is a Mycobacterium spp. In another exemplary embodiment, the bacterium is Mycobacterium avium. In another exemplary embodiment, the bacterium is Mycobacterium avium-intracellulare. In another exemplary embodiment, the bacterium is Mycobacterium kansasii. In another exemplary embodiment, the bacterium is Mycobacterium leprae. In another exemplary embodiment, the bacterium is Mycobacterium lepromatosis. In another exemplary embodiment, the bacterium is Mycobacterium africanum. In another exemplary embodiment, the bacterium is Mycobacterium canetti. In another exemplary embodiment, the bacterium is Mycobacterium microti. In another exemplary embodiment, the bacterium is Mycobacterium tuberculosis. In another exemplary embodiment, the bacterium is Mycobacterium tuberculosis which is multi-drug resistant. In another exemplary embodiment, the bacterium is Mycobacterium tuberculosis which is extensively drug resistant. In another exemplary embodiment, the bacterium is Mycobacterium tuberculosis which is resistant to rifampicin. In another exemplary embodiment, the bacterium is Mycobacterium tuberculosis which is resistant to isoniazid. In another exemplary embodiment, the bacterium is Mycobacterium tuberculosis which is resistant to kanamycin. In another exemplary embodiment, the bacterium is Mycobacterium tuberculosis which is resistant to capreomycin. In another exemplary embodiment, the bacterium is Mycobacterium tuberculosis which is resistant to amikacin.

In another exemplary embodiment, the bacterium is a Pseudomonas species. In another exemplary embodiment, the bacterium is Pseudomonas aeruginosa. In another exemplary embodiment, the bacterium is selected from the group consisting of Pseudomonas aeruginosa, Acinetobacter baumannii, Stenotrophomonas maltophilia and Burkholderia cepacia. In another exemplary embodiment, the bacterium is Acinetobacter baumannii. In another exemplary embodiment, the bacterium is Stenotrophomonas maltophilia. In another exemplary embodiment, the bacterium is Burkholderia cepacia. In another exemplary embodiment, the bacterium is Acinetobacter species. In another exemplary embodiment, the bacterium is Acinetobacter anitratus. In another exemplary embodiment, the bacterium is selected from the group consisting of Enterobacter aerogenes, Enterobacter cloacae, Enterobacter sakazakii, E. coli, K. pneumoniae, P. mirabilis, Serratia marcescens, Citrobacter freundii and Providencia spp. In another exemplary embodiment, the bacterium is selected from the group consisting of Enterobacter aerogenes, Enterobacter cloacae, Enterobacter sakazakii, E. coli, K. pneumoniae, P. mirabilis, Serratia marcescens, Citrobacter freundii, Providencia spp., S. aureus, S. pneumonia, S. pyogenes, E. faecalis, and E. faecium. In another exemplary embodiment, the bacterium is selected from the group consisting of Pseudomonas aeruginosa, Acinetobacter baumannii, Stenotrophomonas maltophilia, Burkholderia cepacia. In another exemplary embodiment, the bacterium is selected from the group consisting of S. aureus, S. pneumonia, S. pyogenes, E. faecalis, and E. faecium. In another exemplary embodiment, the bacterium is selected from the group consisting of Viridans group Strep. In another exemplary embodiment, the bacterium is selected from the group consisting of Strep. mitis, Strep. mutans, Strep. oralis, Strep. sanguis, Strep. sobrinus and Strep. millari.

In another exemplary embodiment, the bacterium is S. pneumonia. In another exemplary embodiment, the bacterium is H. influenzae. In another exemplary embodiment, the bacterium is S. aureus. In another exemplary embodiment, the bacterium is M. catarrhalis. In another exemplary embodiment, the bacterium is M. pneumoniae. In another exemplary embodiment, the bacterium is L. pneumoniae. In another exemplary embodiment, the bacterium is C. pneumoniae. In another exemplary embodiment, the bacterium is S. pyogenes. In another exemplary embodiment, the bacterium is an anaerobe. In another exemplary embodiment, the bacterium is an Alcaligenes species. In another exemplary embodiment, the bacterium is a B. cepacia. In another exemplary embodiment, the bacterium is selected from the group consisting of Enterobacter cloacae, Escherichia coli, Klebsiella pneumoniae, Proteus mirabilis, Providencia stuartii, Serratia marcescens, and Citrobacter freundii. In another exemplary embodiment, the bacterium is resistant to methicillin. In another exemplary embodiment, the bacterium is methicillin-resistant Staphylococcus aureus. In another exemplary embodiment, the bacterium is selected from the group consisting of Streptococcus pneumoniae, Haemophilus influenzae, Staphylococcus aureus, Mycobacterium catarrhalis, Mycobacterium pneumoniae, Legionella pneumophila and Chlamydia pneumoniae. In another exemplary embodiment, the bacterium is selected from the group consisting of Enterobacter cloacae, Escherichia coli, Klebsiella pneumoniae, Proteus mirabilis, Serratia marcescens, Citrobacter freundii, Providencia stuartii, Pseudomonas aeruginosa, Acinetobacter baumannii, Stenotrophomonas maltophilia, Burkholderia cepacia, Staphylococcus aureus, Streptococcus pneumoniae, Streptococcus pyogenes, Enterococcus faecalis, and Enterococcus faecium. In another exemplary embodiment, the bacterium is selected from the group consisting of Staphylococcus aureus, Staphylococcus epidermidis, Staphylococcus haemolyticus, Streptococcus pyogenes, Streptococcus agalactiae and Streptococcus pneumoniae. In one embodiment, the bacterium is Pseudomonas aeruginosa.

In an exemplary embodiment, the bacterium is selected from the group consisting of bacilli, including Bacillus species, Corynebacterium species (also Propionibacterium) and Clostridium species; filamentous bacteria, including Actinomyces species and Streptomyces species; bacilli, such as Pseudomonas species, Brucella species, Agrobacterium species, Bordetella species, Escherichia species, Shigella species, Yersinia species, Salmonella species, Klebsiella species, Enterobacter species, Haemophilus species, Pasteurella species, and Streptobacillus species; spirochetal species, Campylobacter species, Vibrio species; and intracellular bacteria including Rickettsiae species and Chlamydia species.

In another aspect, the invention provides a method of treating and/or preventing a disease. In an exemplary embodiment, the method includes administering to the subject a therapeutically effective amount of a compound of the invention, thereby treating and/or preventing the disease. In an exemplary embodiment, the compound of the invention can be used in human or veterinary medical therapy, particularly in the treatment or prophylaxis of bacterial-associated disease. In an exemplary embodiment, the compound is described herein, or a salt, prodrug, hydrate or solvate thereof, or a combination thereof. In an exemplary embodiment, the invention provides a compound described herein, or a prodrug thereof. In an exemplary embodiment, the invention provides a compound described herein, or a salt, hydrate or solvate thereof. In an exemplary embodiment, the invention provides a compound described herein, or a salt thereof. In another exemplary embodiment, the compound of the invention is a compound described herein, or a pharmaceutically acceptable salt thereof. In an exemplary embodiment, the compound is a compound described herein, or a pharmaceutically acceptable salt thereof. In an exemplary embodiment, the compound is according to a formula described herein, or a pharmaceutically acceptable salt thereof. In an exemplary embodiment, the compound is part of a combination described herein. In an exemplary embodiment, the compound is part of a pharmaceutical formulation described herein. In another exemplary embodiment, the disease is a systemic disease. In another exemplary embodiment, the disease is a topical disease. In an exemplary embodiment, the subject is being administered the compound is not otherwise in need of treatment with the compound.

In an exemplary embodiment, the disease is treated through oral administration of a compound of the invention. In an exemplary embodiment, the disease is treated through intravenous administration of a compound of the invention. In an exemplary embodiment, the disease is treated through subcutaneous administration of a compound of the invention and/or a combination of the invention.

In another aspect, the invention provides a method of treating a systemic disease. The method involves contacting an animal with a compound of the invention and/or a combination of the invention.

In another exemplary embodiment, the disease is associated with a bacteria described herein. In another exemplary embodiment, the disease is associated with infection by a Gram-positive bacteria. In an exemplary embodiment, the disease is associated with a Staphylococcus species. In another exemplary embodiment, the disease is selected from the group consisting of pneumonia, gastroenteritis, toxic shock syndrome, community acquired pneumonia (CAP), meningitis, septic arthritis, urinary tract infection, bacteremia, endocarditis, osteomylitis, skin and skin-structure infection. In an exemplary embodiment, the disease is associated with a Streptococcus species. In another exemplary embodiment, the disease is selected from the group consisting of strep throat, skin infections, necrotizing fasciitis, toxic shock syndrome, pneumonia, otitis media and sinusitis. In an exemplary embodiment, the disease is associated with an Actinomyces species. In another exemplary embodiment, the disease is actinomycosis. In an exemplary embodiment, the disease is associated with a Nocardia species. In another exemplary embodiment, the disease is pneumonia. In one exemplary embodiment, the disease is acute pneumonia. In an exemplary embodiment, the disease is associated with a Corynebacterium species. In another exemplary embodiment, the disease is diphtheria. In an exemplary embodiment, the disease is associated with a Listeria species. In another exemplary embodiment, the disease is meningitis. In an exemplary embodiment, the disease is associated with a Bacillus species. In another exemplary embodiment, the disease is anthrax or food poisoning. In an exemplary embodiment, the disease is associated with a Clostridium species. In another exemplary embodiment, the disease is selected from the group consisting of botulism, tetanus, gas gangrene and diarrhea.

In an exemplary embodiment, the disease is associated with a Mycobacterium species. In an exemplary embodiment, the disease is associated with Mycobacterium tuberculosis. In an exemplary embodiment, the disease is associated with Mycobacterium kansasii. In an exemplary embodiment, the disease is associated with Mycobacterium avium-intracellulare. In another exemplary embodiment, the disease is leprosy. In another exemplary embodiment, the disease is tuberculosis. In another exemplary embodiment, the disease is pulmonary tuberculosis. In another exemplary embodiment, the disease is extrapulmonary tuberculosis. In another exemplary embodiment, the disease is associated with multi-drug resistant tuberculosis. In another exemplary embodiment, the disease is associated with extensively drug resistant tuberculosis.

In another exemplary embodiment, the disease is associated with infection by a Gram-negative bacteria. In an exemplary embodiment, the disease is associated with a Neisseria species. In another exemplary embodiment, the disease is selected from the group consisting of meningitis, gonorrhea, otitis extema and folliculitis. In an exemplary embodiment, the disease is associated with an Escherichia species. In another exemplary embodiment, the disease is selected from the group consisting of diarrhea, urinary tract infections, meningitis, sepsis and HAP. In an exemplary embodiment, the disease is associated with a Shigella species. In another exemplary embodiment, the disease is selected from the group consisting of diarrhea, bacteremia, endocarditis, meningitis and gastroenteritis. In an exemplary embodiment, the disease is associated with a Salmonella species. In another exemplary embodiment, the disease is selected from the group consisting of Typhoid fever, sepsis, gastroenteritis, endocarditis, sinusitis and meningitis. In an exemplary embodiment, the disease is associated with a Yersinia species. In another exemplary embodiment, the disease is selected from the group consisting of Typhoid fever, bubonic plague, enteric fever and gastroenteritis. In an exemplary embodiment, the disease is associated with a Klebsiella species. In another exemplary embodiment, the disease is sepsis or urinary tract infection. In an exemplary embodiment, the disease is associated with a Proteus species. In another exemplary embodiment, the disease is an urinary tract infection. In an exemplary embodiment, the disease is associated with an Enterobacter species. In another exemplary embodiment, the disease is a hospital-acquired infection. In an exemplary embodiment, the disease is associated with a Serratia species. In another exemplary embodiment, the disease is selected from the group consisting of a urinary tract infection, skin and skin-structure infection and pneumonia. In an exemplary embodiment, the disease is associated with a Vibrio species. In another exemplary embodiment, the disease is cholera or gastroenteritis. In an exemplary embodiment, the disease is associated with a Campylobacter species. In another exemplary embodiment, the disease is gastroenteritis. In an exemplary embodiment, the disease is associated with a Helicobacter species. In another exemplary embodiment, the disease is chronic gastritis. In an exemplary embodiment, the disease is associated with a Pseudomonas species. In another exemplary embodiment, the disease is selected from the group consisting of pneumonia, osteomylitis, burn-wound infections, sepsis, UTIs, endocarditis, otitis and corneal infections. In an exemplary embodiment, the disease is associated with a Bacteroides species. In another exemplary embodiment, the disease is periodontal disease or aspiration pneumonia. In an exemplary embodiment, the disease is associated with a Haemophilus species. In another exemplary embodiment, the disease is selected from the group consisting of meningitis, epiglottitis, septic arthritis, sepsis, chancroid and vaginitis. In an exemplary embodiment, the disease is associated with a Bordetella species. In another exemplary embodiment, the disease is Whooping cough. In an exemplary embodiment, the disease is associated with a Legionella species. In another exemplary embodiment, the disease is pneumonia or pontiac fever. In an exemplary embodiment, the disease is associated with a Francisella species. In another exemplary embodiment, the disease is tularemia. In an exemplary embodiment, the disease is associated with a Brucella species. In another exemplary embodiment, the disease is brucellosis. In an exemplary embodiment, the disease is associated with a Pasteurella species. In another exemplary embodiment, the disease is a skin infection. In an exemplary embodiment, the disease is associated with a Gardnerella species. In another exemplary embodiment, the disease is vaginitis. In an exemplary embodiment, the disease is associated with a Spirochetes species. In another exemplary embodiment, the disease is syphilis or Lyme disease. In an exemplary embodiment, the disease is associated with a Chlamydia species. In another exemplary embodiment, the disease is chlamydia. In an exemplary embodiment, the disease is associated with a Rickettsiae species. In another exemplary embodiment, the disease is Rocky Mountain spotted fever or typhus.

In an exemplary embodiment, the disease is associated with Mycoplasma pneumoniae. In another exemplary embodiment, the disease is tracheobronchitis or walking pneumonia. In an exemplary embodiment, the disease is associated with Ureaplasma urealyticum. In another exemplary embodiment, the disease is urethritis. In another exemplary embodiment, the disease is pyelonephritis. In another exemplary embodiment, the disease is an intra-abdominal infection. In another exemplary embodiment, the disease is febrile neutropenia. In another exemplary embodiment, the disease is a pelvic infection. In another exemplary embodiment, the disease is bacteraemia. In another exemplary embodiment, the disease is septicaemia.

In an exemplary embodiment, the disease is an acute exacerbation of chronic obstructive pulmonary disease. In an exemplary embodiment, the disease is chronic obstructive pulmonary disease. In an exemplary embodiment, the disease is pharyngitis. In an exemplary embodiment, the disease is tonsillitis. In an exemplary embodiment, the disease is Acute Exacerbation of Chronic Bronchitis (AECB). In an exemplary embodiment, the disease is cervicitis. In an exemplary embodiment, the disease is genital ulcer disease.

In an exemplary embodiment, the disease is a Gram-negative bacterial infection. In an exemplary embodiment, the Gram-negative bacteria is Pseudomonas. In an exemplary embodiment, the Gram-negative bacteria is Pseudomonas aeruginosa.

In an exemplary embodiment, the Gram-negative bacterial infection is pneumonia. In an exemplary embodiment, the Gram-negative bacterial infection is acute pneumonia.

In an exemplary embodiment, for any of the methods described herein, the subject is an animal. In an exemplary embodiment, the animal is selected from the group consisting of human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, for any of the methods described herein, the animal is selected from the group consisting of a human, cattle, goat, pig, sheep, horse, cow, bull, dog, guinea pig, gerbil, rabbit, cat, chicken and turkey. In another exemplary embodiment, for any of the methods described herein, the subject or the animal is a human. In an exemplary embodiment, for any of the methods described herein, a compound described herein or a pharmaceutically acceptable salt thereof, and/or a pharmaceutical formulation described herein can be used. In some embodiments, the compound is a tanshinone or tanshinone analog. In some embodiments, the compound is a compound identified by the screening methods as described herein.

Efficacy of the methods, compounds, and combinations of compounds described herein in treating, preventing and/or managing the indicated diseases or disorders can be tested using various animal models known in the art.

### Pharmaceutical Compositions

In an embodiment, the invention provides a pharmaceutical composition for use in the treatment of the diseases and conditions described herein.

In one aspect, a pharmaceutical composition for the treatment or prevention of a bacterial infection in a subject in need thereof, is provided, the composition comprising an inhibitor of Type 3 Secretion System (T3SS). In some embodiments, the T3SS inhibitor is tanshinone 1 (TSN1), dihydrotanshinone 1 (dHTSN1), or dihydrotanshinone (dHTSN).

In one aspect, a pharmaceutical composition for the treatment or prevention of a bacterial infection in a subject in need thereof, is provided, the composition comprising an inhibitor of Type 3 Secretion System (T3SS), and a pharmaceutically acceptable carrier, wherein the inhibitor of T3SS blocks interaction between a T3SS needle protein and a T3SS chaperone protein. In another aspect, a pharmaceutical composition for the treatment or prevention of a bacterial infection in a subject in need thereof is provided, the composition comprising an agent identified as an inhibitor of Type 3 Secretion System (T3SS) according to the methods of identifying T3SS inhibitors describe herein, and a pharmaceutically acceptable carrier. In some embodiments, the bacterial infection is a Gram-negative bacterial infection.

In still another aspect, a pharmaceutical composition for the treatment of a Gram-negative bacterial infection in a subject in need thereof, the composition comprising an inhibitor of Type 3 Secretion System (T3SS) selected from the group consisting of tanshinone 1 (TSN1), dihydrotanshinone 1 (dHTSN1), dihydrotanshinone (dHTSN), and the pharmaceutically acceptable salts, solvates, hydrates, cocrystals, or prodrugs thereof; and a pharmaceutically acceptable carrier.

The pharmaceutical compositions are typically formulated to provide a therapeutically effective amount of a compound, as described herein, or a pharmaceutically acceptable salt, solvate, or hydrate thereof, as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are preferably for use in the treatment of bacterial infection.

In some embodiments, the concentration of a compound described herein (e.g., tanshinone or tanshinone analog compound), or pharmaceutically acceptable salt thereof, provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of a compound as described herein or pharmaceutically acceptable salt thereof provided in the pharmaceutical compositions of the invention is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of a compound as described herein or pharmaceutically acceptable salt thereof, provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of a compound as described herein or pharmaceutically acceptable salt thereof, provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of a compound described herein (e.g., T3SS inhibitors, including tanshinone and tanshinone analogs), or pharmaceutically acceptable salt thereof, provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of: a compound described herein (e.g., T3SS inhibitors, including tanshinone and tanshinone analogs), or pharmaceutically acceptable salt thereof, provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the invention is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the invention provides a pharmaceutical composition for oral administration containing: a compound described herein (e.g., T3SS inhibitors such as tanshinone and tanshinone analogs, including tanshinone 1 (TSN1), dihydrotanshinone 1 (dHTSN1) or dihydrotanshinone (dHTSN)), or pharmaceutically acceptable salt thereof, described herein, and a pharmaceutical excipient suitable for administration.

In preferred embodiments, the invention provides a solid pharmaceutical composition for oral administration containing: (i) an effective amount of: a tanshinone or tanshinone analog, or pharmaceutically acceptable salt thereof, and (ii) a pharmaceutical excipient suitable for administration. In some embodiments, the composition further contains (iii) an effective amount of an additional active pharmaceutical ingredient. For example, additional active pharmaceutical ingredients, as used herein, may include one or more compounds that kill a bacteria.

In some embodiments, the pharmaceutical composition may be a liquid pharmaceutical composition suitable for oral consumption.

Pharmaceutical compositions of the invention suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present invention and to minimize precipitation of the compound of the present invention. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the invention provides a pharmaceutical composition for injection containing: a tanshinone or tanshinone analog, or pharmaceutically acceptable salt thereof, described herein, and a pharmaceutical excipient suitable for injection. Components and amounts of compounds in the compositions are as described herein.

The forms in which the compositions of the invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating: a tanshinone or tanshinone analog, or pharmaceutically acceptable salt thereof, described herein, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the invention provides a pharmaceutical composition for transdermal delivery containing: a compound described herein (e.g., T3SS inhibitors such as tanshinone and tanshinone analogs, including tanshinone 1 (TSN1), dihydrotanshinone 1 (dHTSN1) or dihydrotanshinone (dHTSN)), or pharmaceutically acceptable salt thereof, described herein, and a pharmaceutical excipient suitable for transdermal delivery.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of: a compound described herein (e.g., T3SS inhibitors such as tanshinone and tanshinone analogs, including tanshinone 1 (TSN1), dihydrotanshinone 1 (dHTSN1) or dihydrotanshinone (dHTSN)), or pharmaceutically acceptable salt thereof, described herein, in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of: a compound described herein (e.g., T3SS inhibitors such as tanshinone and tanshinone analogs, including tanshinone 1 (TSN1), dihydrotanshinone 1 (dHTSN1) or dihydrotanshinone (dHTSN)), or pharmaceutically acceptable salt thereof, described herein, or a pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, can also be administered intraadiposally or intrathecally.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. A compound (e.g., T3SS inhibitors such as tanshinone and tanshinone analogs, including tanshinone 1 (TSN1), dihydrotanshinone 1 (dHTSN1) or dihydrotanshinone (dHTSN)), or pharmaceutically acceptable salt thereof, described herein, may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. A compound (e.g., T3SS inhibitors such as tanshinone and tanshinone analogs, including tanshinone 1 (TSN1), dihydrotanshinone 1 (dHTSN1) or dihydrotanshinone (dHTSN)), or pharmaceutically acceptable salt thereof, described herein, may additionally be administered intravascularly from a balloon used during angioplasty. Extravascular administration of a compound (e.g., T3SS inhibitors such as tanshinone and tanshinone analogs, including tanshinone 1 (TSN1), dihydrotanshinone 1 (dHTSN1) or dihydrotanshinone (dHTSN)), or pharmaceutically acceptable salt thereof, described herein, via the pericard or via advential application of formulations of the invention may also be performed to decrease restenosis.

Exemplary parenteral administration forms include solutions or suspensions of a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, in sterile aqueous solutions, for example, aqueous propylene glycol or dextrose solutions. Such dosage forms can be suitably buffered, if desired.

The invention also provides kits. The kits include a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In some embodiments, the compound described herein (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, and another active pharmaceutical ingredient are provided as separate compositions in separate containers within the kit. In some embodiments, the compound described herein (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In a preferred embodiment, the kits are for use in the treatment of bacterial infection.

In a particular embodiment, the kits described herein are for use in the treatment of bacterial infection. In some embodiments, the kits described herein are for use in the treatment of a bacterial infection caused by and/or associate with a Gram-negative bacteria. In some embodiments, the kits described herein are for use in the treatment of a bacterial infection selected from the group consisting of bacterial infection is a lung infection, skin infection, soft tissue infection, gastrointestinal infection, urinary tract infection, meningitis, or sepsis. In some embodiments, the bacterial infection is pneumonia. In some embodiments, the bacterial infection is acute pneumonia.

### Dosages and Dosing Regimens

The amounts of: a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, may be provided in units of mg/kg of body mass or in mg/m² of body surface area.

In some embodiments, a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein is administered in multiple doses. In a preferred embodiment, a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein is administered in multiple doses. Dosing may be once, twice, three times, four times, five times, six times, or more than six times per day. Dosing may be once a month, once every two weeks, once a week, or once every other day. In other embodiments, a compound (e.g., T3 SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, is administered about once per day to about 6 times per day. In some embodiments, a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, is administered once daily, while in other embodiments, a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein is administered twice daily, and in other embodiments a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, is administered three times daily.

Administration of a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, may continue as long as necessary. In some embodiments, a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, is administered for more than 1, 2, 3, 4, 5, 6, 7, 14, or 28 days. In some embodiments, a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein is administered for less than 28, 14, 7, 6, 5, 4, 3, 2, or 1 day. In some embodiments, a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein is administered chronically on an ongoing basis—e.g., for the treatment of chronic effects. In another embodiment, the administration of a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, continues for less than about 7 days. In yet another embodiment, the administration continues for more than about 6, 10, 14, 28 days, two months, six months, or one year. In some cases, continuous dosing is achieved and maintained as long as necessary.

In some embodiments, an effective dosage of a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage of a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of a compound (e.g., T3SS inhibitors such as tanshinone and tanshinone analogs, including tanshinone 1 (TSN1), dihydrotanshinone 1 (dHTSN1) and dihydrotanshinone (dHTSN)), or pharmaceutically acceptable salt thereof, described herein, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### Methods of Inhibiting Biogenesis of Type 3 Secretion System Needle

In one aspect, there is provided a method of inhibiting treating or preventing bacterial infection in a subject in need thereof, the method comprising administering to the subject a therapeutically effective amount of an inhibitor of Type 3 Secretion System (T3SS), wherein the inhibitor of T3SS blocks interaction between a T3SS needle protein and a T3SS chaperone protein.

In another aspect, a method of inhibiting Type 3 Secretion System (T3SS) is provided, the method comprising contacting a T3SS needle protein or a T3SS chaperone protein with an agent that blocks interaction between the T3SS needle protein and T3SS chaperone protein. In another aspect, a method of inhibiting biogenesis of a Type 3 Secretion System (T3SS) needle is provided, the method comprising contacting a T3SS needle protein or a T3SS chaperone protein with an agent that blocks interaction between the T3SS needle protein and T3SS chaperone protein.

In another aspect, a method of reducing bacterial virulence, cytotoxicity, and/or pathogenicity is provided, the method comprising contacting a T3SS needle protein or a T3SS chaperone protein with an agent that blocks interaction between the T3SS needle protein and T3SS chaperone protein. In another aspect, a method of reducing secretion of a bacterial virulence factor is provided, the method comprising contacting a T3SS needle protein or a T3SS chaperone protein with an agent that blocks interaction between the T3SS needle protein and T3SS chaperone protein. In some embodiments, the blocking of the interaction between the T3SS needle protein and T3SS chaperone protein inhibits assembly or biogenesis of the T3SS needle protein.

In some embodiments, the T3SS needle protein is PscF and the T3SS chaperone protein is PscE-PscG. In some embodiments, the agent that blocks interaction competes for binding to the T3SS needle protein with a T3SS chaperone protein, or competes for binding to a T3SS chaperone protein with a T3SS needle protein. In some embodiments, the agent competes for binding with an IC50 less than 5 μM, less than 4 μM, less than 3 μM, less than 2 μM, or less than 1 μM. In some embodiments, the agent competes for binding with an IC50 between 0.1 μM to 5 μM, between 0.3 μM to 5 μM, between 0.5 μM to 3 μM, between 0.5 μM to 2 μM, between 0.5 μM to 1 μM, between 0.1 μM to 3 μM, between 0.1 μM to 1 μM, between 0.3 μM to 3 μM, between 0.3 μM to 2 μM. In some embodiments, the agent competes for binding with an IC50 of about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 1.5 μM, about 2 μM, about 2.5 μM, about 3 μM, about 3.5 μM, about 4 μM, about 4.5 μM, or about 5 μM.

In some embodiments, the agent binds a PscF-binding pocket of PscG. In some embodiments, the agent binds one or more residues on PscG selected from Trp79, Trp 67, Trp73, and Trp31. In some embodiments, the agent binds residue Trp79 on PscG.

In some embodiments, the agent that blocks interaction between the T3SS needle protein and T3SS chaperone protein is a tanshinone or tanshinone analog, or the pharmaceutically acceptable salts, solvates, hydrates, cocrystals, or prodrugs thereof. In some embodiments, the agent that blocks interaction between the T3SS needle protein and T3SS chaperone protein tanshinone 1 (TSN1), dihydrotanshinone 1 (dHTSN1), dihydrotanshinone (dHTSN), or the pharmaceutically acceptable salts, solvates, hydrates, cocrystals, or prodrugs thereof.

### Methods of Screening

As described herein, fluorescence polarization-based screening identified tanshinones as inhibitors of the biogenesis of the type 3 secretion system needle of Pseudomonas aeruginosa with potent antibacterial activity.

In one aspect, there is provided a method of identifying an inhibitor of Type 3 Secretion System (T3SS), the method comprising: (a) adding a candidate agent to a composition comprising a protein complex in the T3SS, wherein a component of the protein complex is fluorescently labeled; (b) determining the fluorescence polarization (FP) of the fluorescently labeled component; wherein the candidate agent is identified as an inhibitor of T3SS if the FP is decreased relative to a reference FP level. In some embodiments, the protein complex comprises a T3SS needle protein and a T3SS chaperone protein. In some embodiments, the protein complex is PscF-PscE-PscG. In some embodiments, the method is high-throughput.

In some embodiments, the PscF is labeled with a fluorescent label. The fluorescent label can be any known in the art, e.g., fluorescent dyes such as fluorescein, rhodamine, or BODIPY.

In some embodiments, the PscF is truncated. In some embodiments, the labeled PscF comprises the amino acid sequence TVTRALRDLMQGILQKI (SEQ ID NO: 1).

In some embodiments, the reference FP level is the FP of the fluorescently labeled component without a candidate agent added to the composition. In some embodiments, the composition is a solution. The candidate agent can be a small molecule compound. In some embodiments, the candidate agent is a tanshinone or tanshinone analog.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Design of Screen for Inhibitors of T3SS Needle Biogenesis

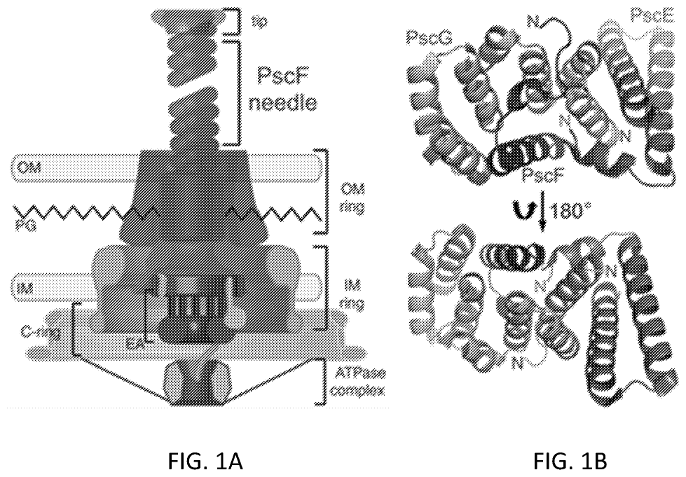

In the Pseudomonas aeruginosa T3SS (FIG. 1A), the needle is formed by multiple copies of a single protein termed PscF of 85 amino acid residues. Prior to its secretion for needle assembly, PscF is protected in a heterotrimeric complex by two chaperone proteins, PscE (67 AA) and PscG (115 AA). PscG, stabilized by PscE, presents a large concaved hydrophobic surface for interactions with the non-polar residues of PscF (FIG. 1B); Pseudomonas aeruginosa mutants deficient in either PscE or PscG or both fail to secrete PscF for assembly of the T3SS needle and, consequently, are non-cytotoxic. Thus, inhibitors that block PscF interactions with the PscE-PscG dimer are expected to induce premature aggregation and degradation of PscF in the bacterial cytosol, debilitating the biogenesis of the Pseudomonas aeruginosa T3SS needle. Since the survival of Pseudomonas aeruginosa is independent of the T3SS, such inhibitors are less likely to induce antibiotic resistance, therefore ideally suited for development as a novel class of therapeutics to treat multidrug-resistant Pseudomonas aeruginosa infections. To facilitate their discovery, a fluorescence polarization (FP)-based high throughput screening (HTS) system was designed and validated, and a surprising discovery made in a proof-of-concept study.

### Example 2: Chemical Synthesis and Characterization of PscE, PscF and PscG

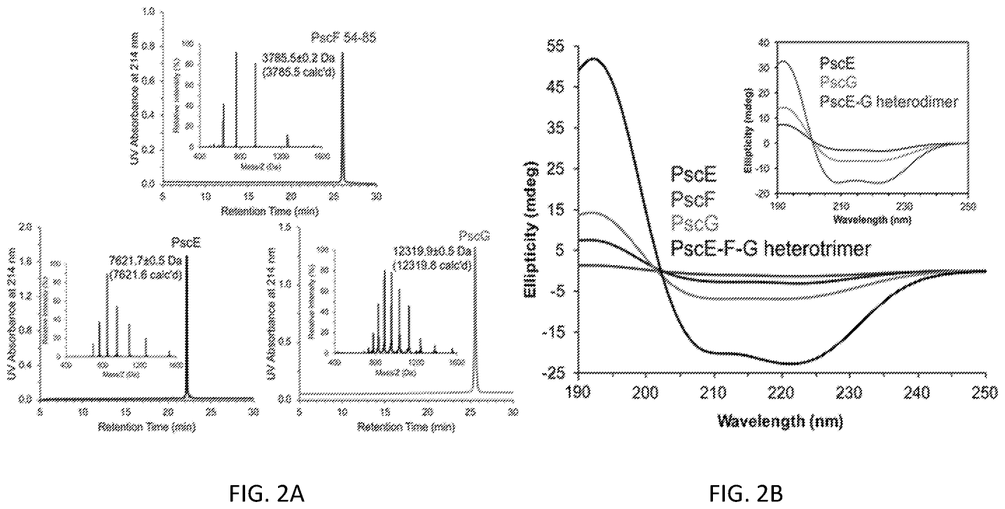

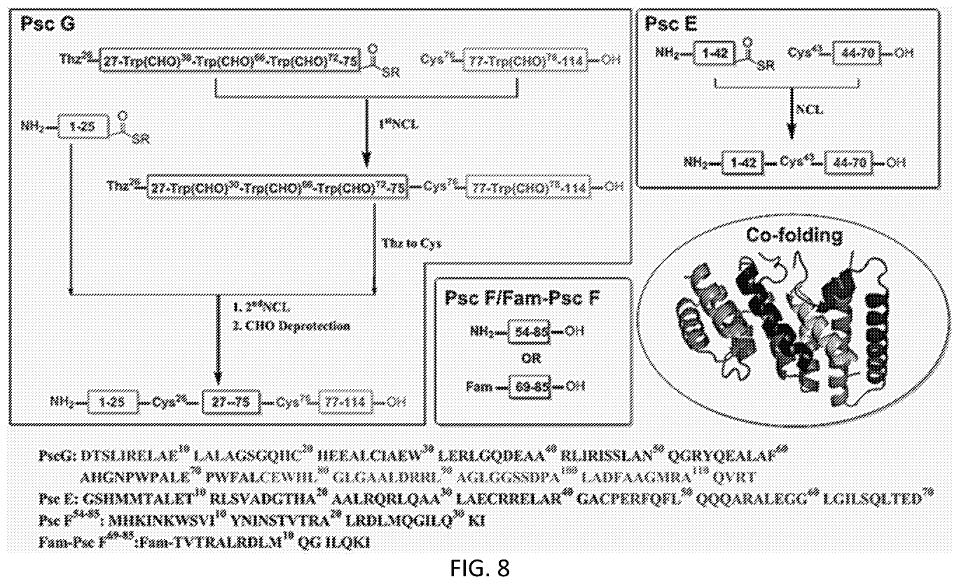

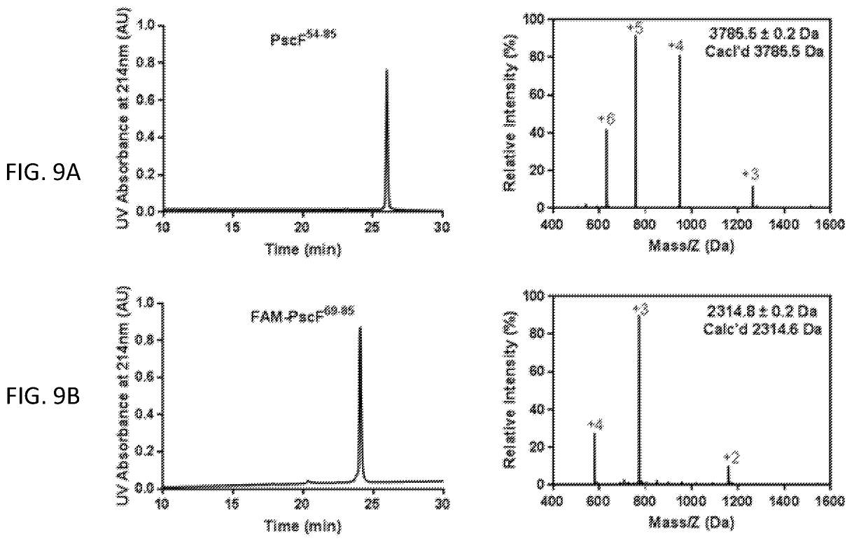

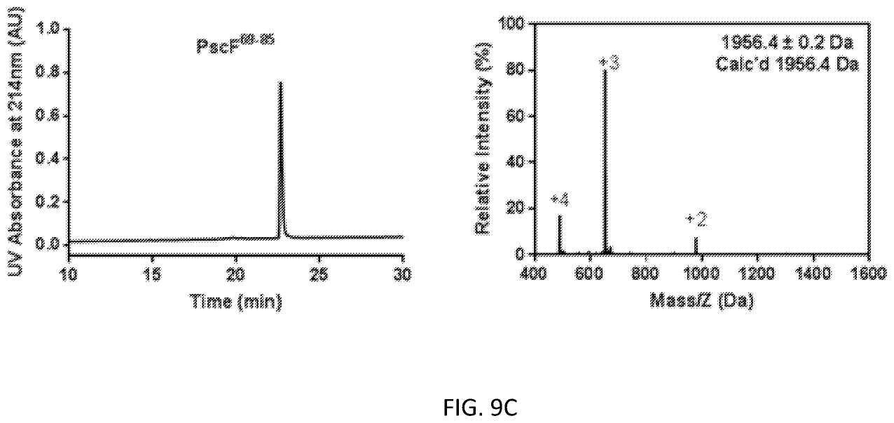

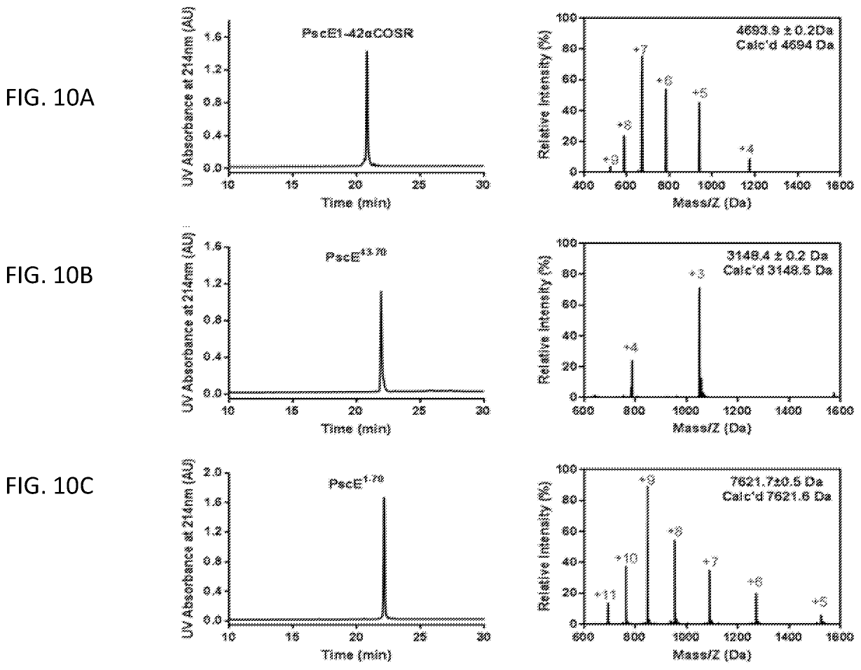

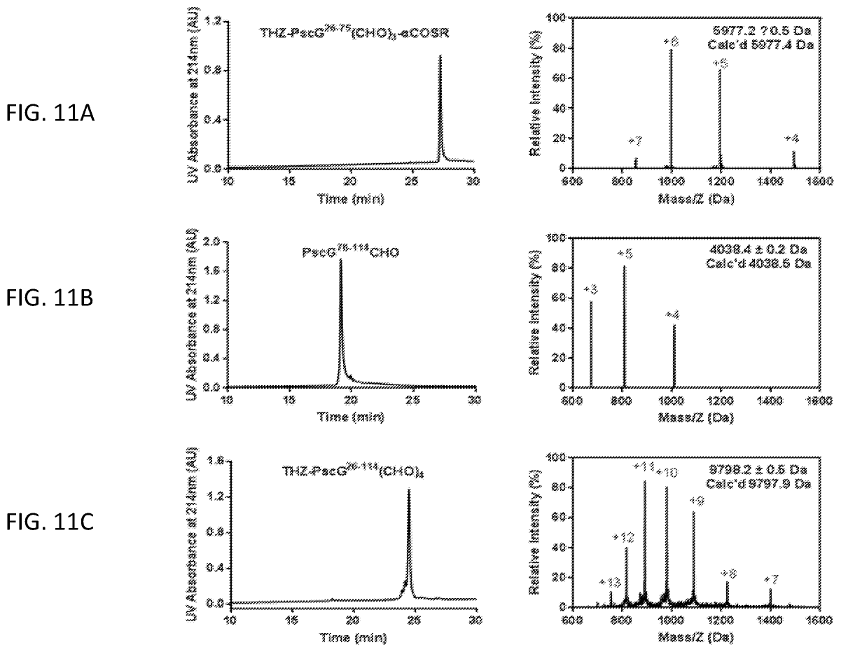

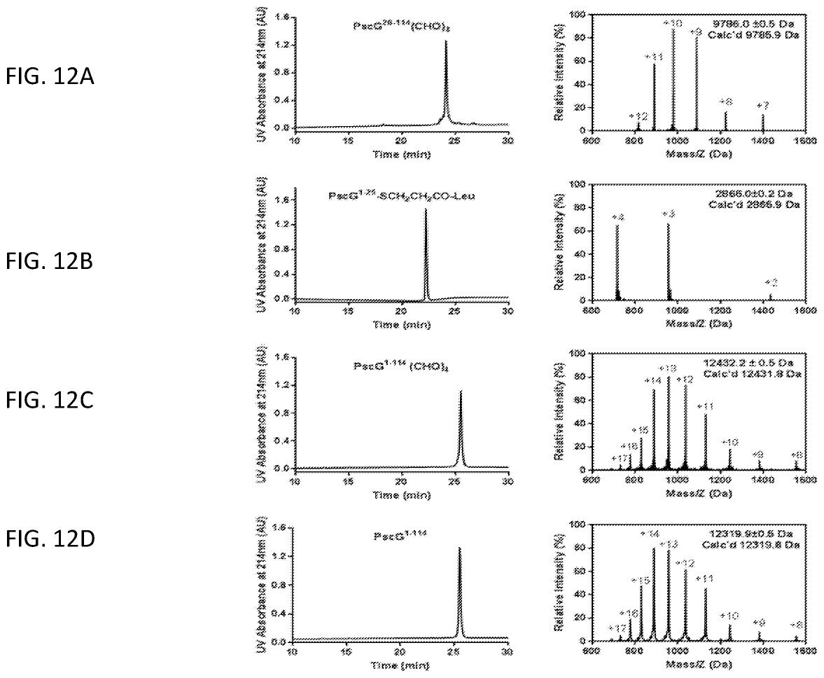

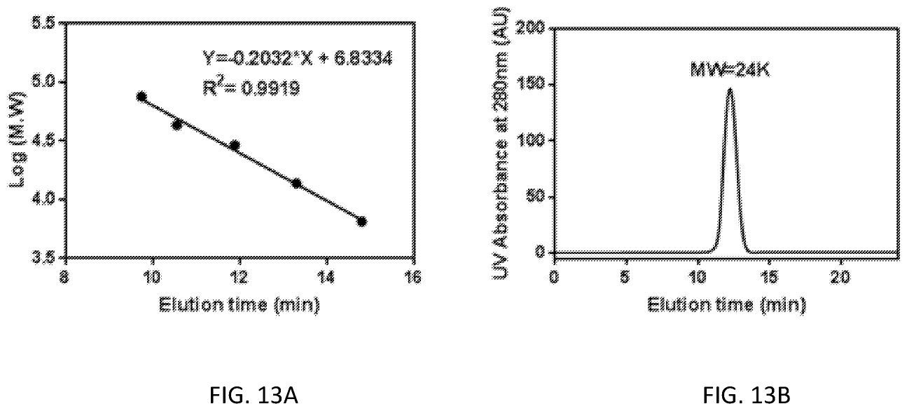

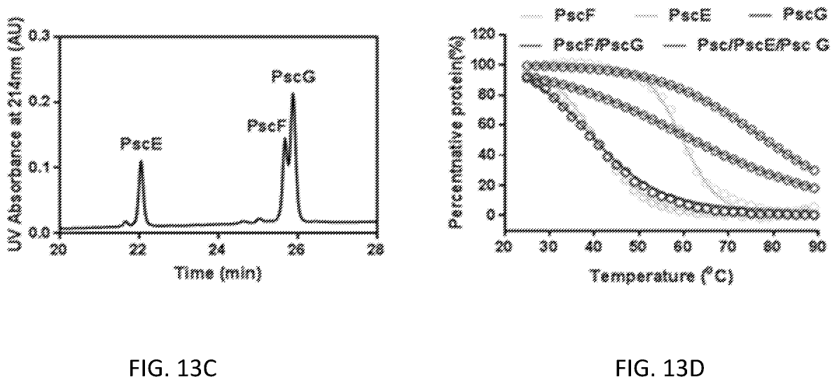

Quinaud et al. reported the first crystal structure of the trimeric complex of PscE-PscF-PscG formed by recombinant proteins. To replicate their findings, PscE, PscG and PscF⁵⁴⁻⁸⁵ were chemically synthesized using solid phase peptide synthesis coupled with native chemical ligation (FIGS. 8-12). (Note: The N-terminally truncated PscF⁵⁴⁻⁸⁵ was prepared because PscF¹⁻⁵³ is structurally disordered and does not contribute to interactions with PscE-PscG (FIG. 1B)). All synthetic polypeptides were purified to homogeneity by reversed phase (RP)-HPLC and verified by electrospray ionization mass spectrometry (ESI-MS) (FIG. 2A). To obtain the heterotrimeric complex previously described, PscE, PscG and PscF⁵⁴⁻⁸⁵ at a 1:1:1 molar ratio were dissolved in 6 M GuHCl, followed by a 6-fold dilution with and an overnight dialysis against PBS. The resultant protein complex was analyzed by Superdex 75 size-exclusion chromatography, RP-HPLC and ESI-MS, and confirmed as a heterotrimer (FIGS. 13A-13C). Consistent with the known structural and biophysical properties of the PscE-PscG-PscF⁵⁴⁻⁸⁵ complex, the synthetic heterotrimer adopted a predominantly alpha-helical conformation in solution as determined by CD spectroscopy (FIG. 2B) and was highly stable as evidenced by a melting temperature of 61.7° C. measured in a protein thermal denaturation assay (FIG. 13D). As was previously reported, it was also found that synthetic PscE and PscG could form a stable heterodimer of a helical nature with a slightly lower melting temperature (FIGS. 2B, 13D).

### Example 3: Design and Validation of an FP-Based Readout for HTS

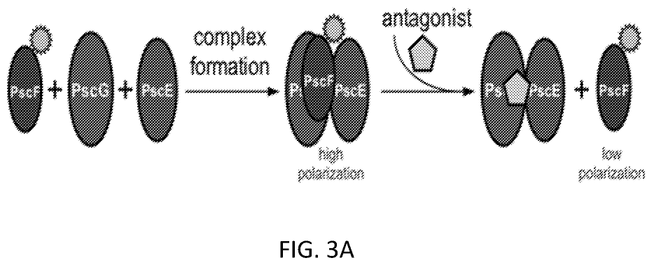

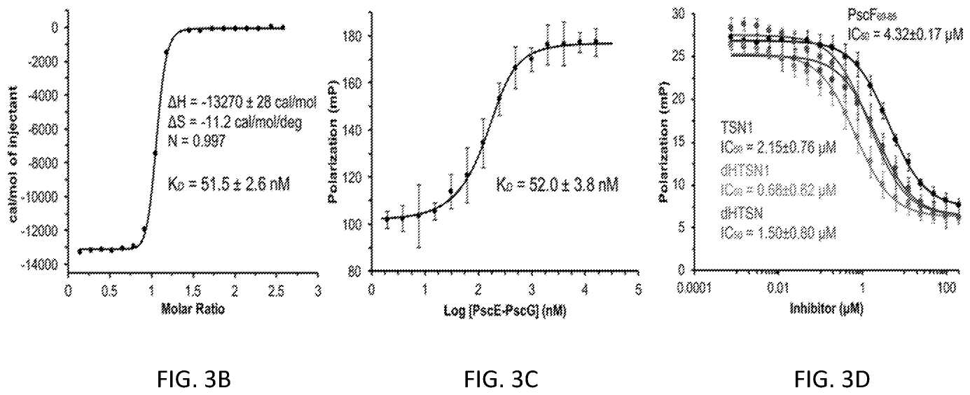

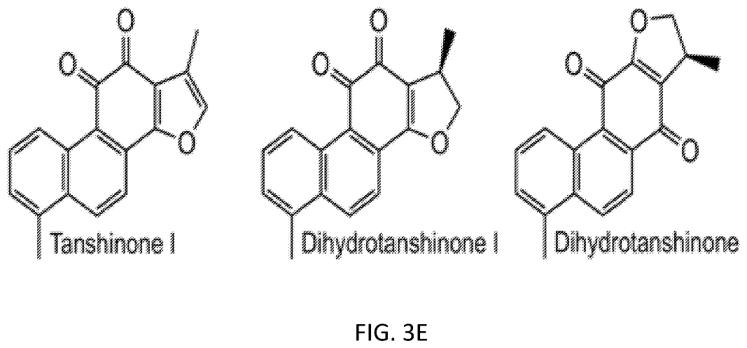

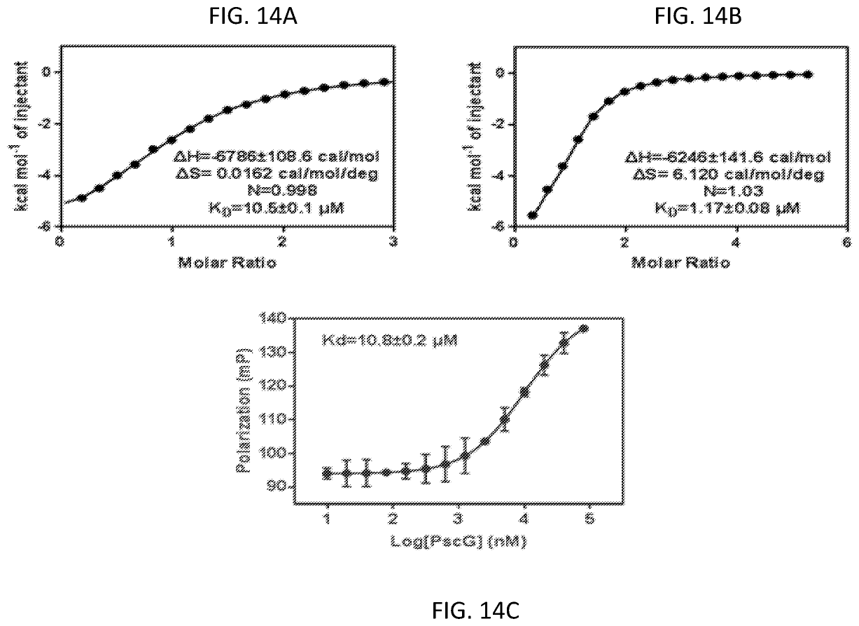

FP assays have been widely used in HTS for low molecular weight inhibitors that target proteins such as enzymes and receptors in the presence of a small, fluorescently labeled natural substrate or ligand of the target protein. For dyes attached to small, rapidly rotating molecules, FP is low as the molecules tumble fast in solution (relative to the fluorescence lifetime) and efficiently “scrambles” the polarization of emitted light. However, upon binding by a large molecule, tumbling of the dye complex is slowed, resulting in an increased polarization of fluorescence emission. The strategy for our FP assay is illustrated in FIG. 3A, where addition of a library compound to the high-polarization heterotrimeric complex leads to the displacement of fluorescently labeled PscF from the PscE-PscG heterodimer, resulting in a decrease in FP. Since PscF⁵⁴⁻⁸⁵ readily precipitated in aqueous solution, the peptide was further truncated by deleting 15 amino acid residues at its N-terminus (FIG. 8), yielding a soluble PscF⁶⁹⁻⁸⁵ peptide, which was subsequently labeled with fluorescein (FAM). Structural studies showed that the interaction of PscF⁵⁴⁻⁸⁵ with PscE-PscG is dominated by the C-terminal amphipathic α-helix PscF⁶⁹⁻⁸⁵ rather than the N-terminal extended coil comprising residues 54-66 (FIG. 1B). For functional verification, though, the interactions of PscE, PscG and unlabeled PscF⁶⁹⁻⁸⁵ were characterized using isothermal titration calorimetry (ITC) (FIGS. 14A-14B). An equilibrium dissociation constant (K_{D}) of 1.17 μM was determined for PscE and PscG. While titration of PscF⁶⁹⁻⁸⁵ to PscG alone yielded a K_{D} value of 10.5 μM, a significantly stronger binding was observed for PscF⁶⁹⁻⁸⁵ interacting with the preformed PscE-PscG dimer (K_{D}=51.5 nM) (FIG. 3B). Nearly identical K_{D} values (10.8 μM and 52.0 nM, respectively) were obtained using ^{FAM}PscF⁶⁹⁻⁸⁵ in an FP assay (FIGS. 3C, 14C). Of note, unlabeled PscF⁶⁹⁻⁸⁵ competed off ^{FAM}PscF⁶⁹⁻⁸⁵ from PscE-PscG in a dose-dependent fashion, giving rise to an IC₅₀ value of 4.32 μM (FIG. 3D). Collectively, these functional data fully validated the synthetic heterotrimeric complex PscE-PscG-^{FAM}PscF⁶⁹⁻⁸⁵ as a suitable system for the development of an FP assay for HTS, and supported the structural finding as well that although PscE does not directly participate in PscF interactions, it enhances them by stabilizing the scaffold of PscG.

### Example 4: Identification of Tanshinones as Competitive Inhibitors of PscF Binding to PscE-PscG

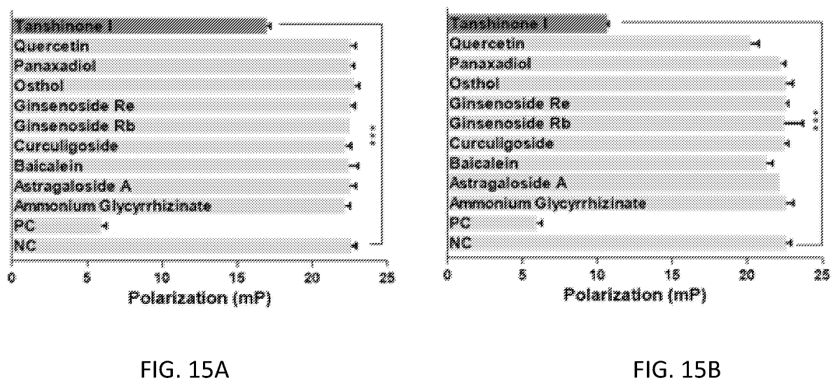

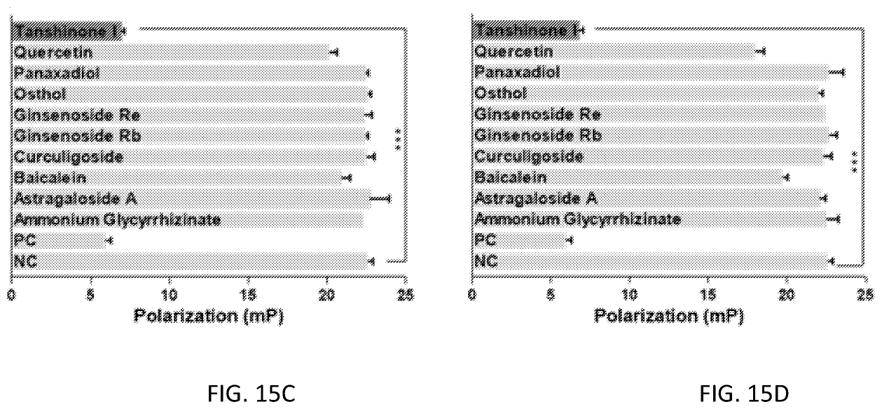

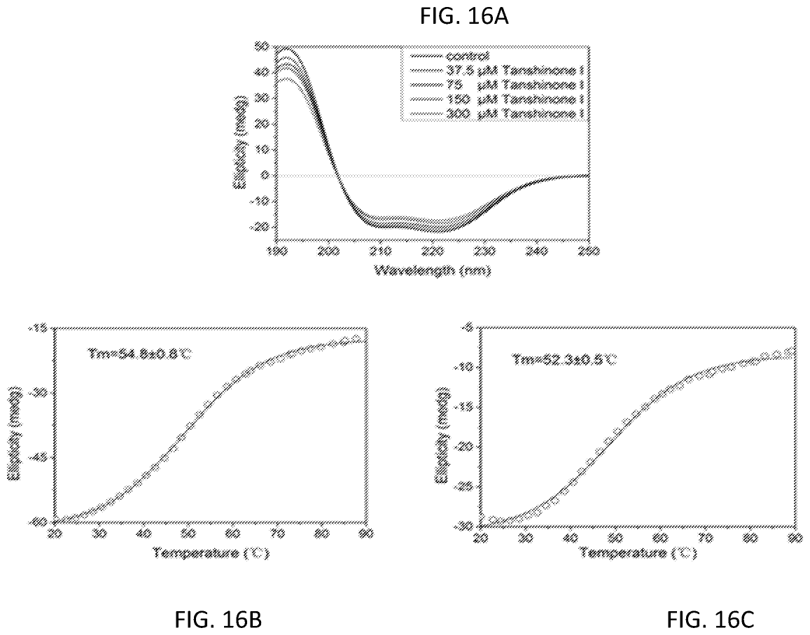

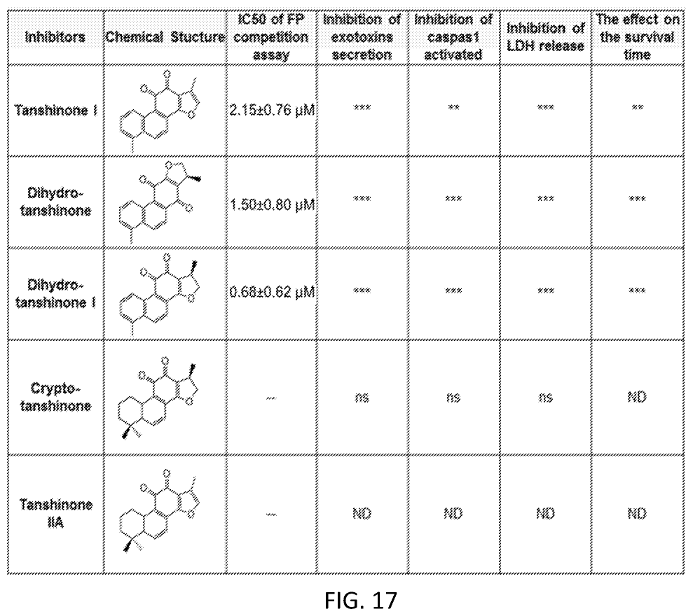

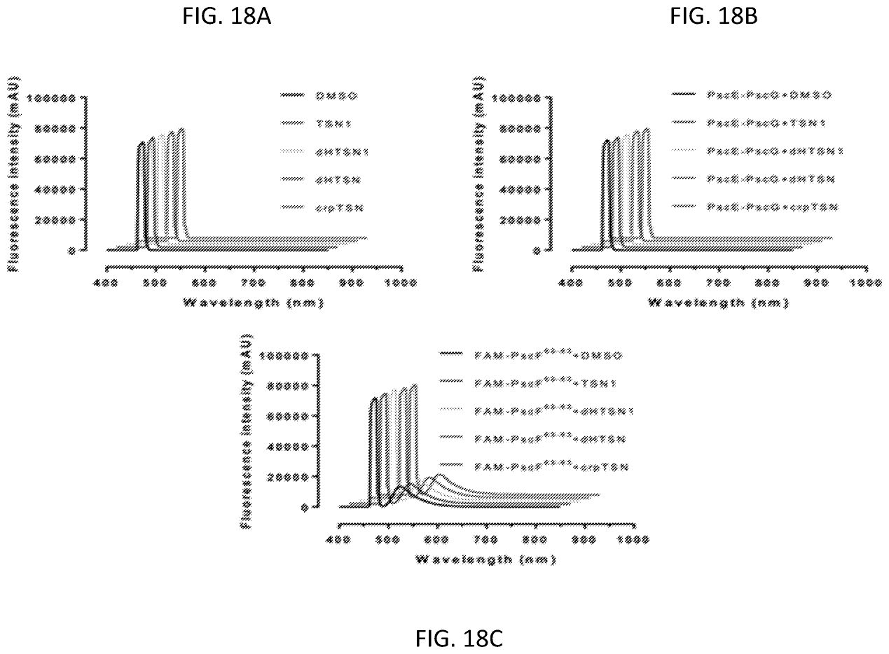

Armed with this validated FP assay, an ultra-low throughput ten natural herbal compounds in traditional Chinese medicine were screened: ammonium glycyrrhizinate, astragaloside A, baicalein, curculigoside, ginsenosides Rb1 and Re, osthol, panaxadiol, quercetin, and tanshinone 1 (TSN1) (FIG. 15). These compounds with various anti-infective, anti-inflammatory or anti-tumor properties but no known activity against bacterial secretion systems were obtained from a collaborator's laboratory and intended initially as “negative controls” for a proof-of-concept study. To our complete surprise, both primary and secondary screenings identified TSN1 as a positive hit, which competed with ^{FAM}PscF⁶⁹⁻⁸⁵ for PscE-PscG binding in an FP assay, yielding an IC₅₀ value of 2.15 μM (FIG. 15, FIGS. 3D-3E). Consistent with this purely serendipitous finding, TSN1, when added to the preformed heterotrimer PscE-PscG-PscF⁶⁹⁻⁸⁵, reduced not only α-helicity of the complex (FIG. 16A), but also its thermal stability (FIGS. 16B-16C). For further verification, four additional commercially available tanshinone analogs (FIG. 17) were examined and dihydrotanshinone 1 (dHTSN1) (IC₅₀=0.68 μM) and dihydrotanshinone (dHTSN) (IC₅₀=1.50 μM) were identified as similar in structure to and more active than TSN1 (FIGS. 3D-3E). By contrast, cryptotanshinone (crpTSN) and tanshinone 2A were inactive (FIG. 17). Of note, fluorophores that excite and emit at longer wavelengths should in general be used for FP-based HTS assays in order to minimize potential spectral interference by library compounds. While FAM was not an ideal choice of fluorophore for this purpose, tanshinones, which are not fluorescent themselves, showed no spectroscopic interference with the FAM fluorescence when excited at 470 nm (FIG. 18).

### Example 5: Structural Characterization of Tanshinone Interactions with PscE-PscG

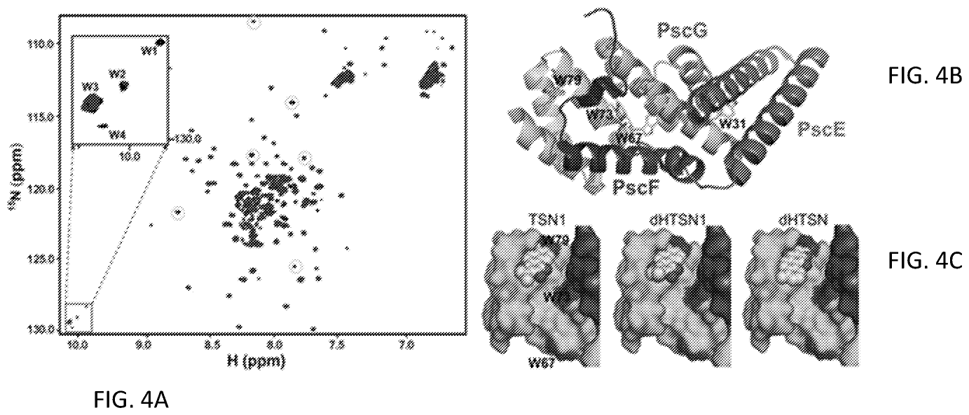

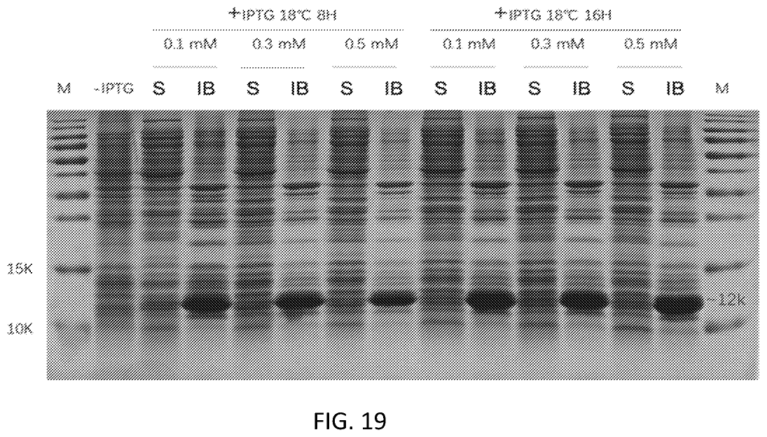

For structural validation, a heterodimeric complex comprising synthetic PscE and an ¹⁵N-labelled recombinant PscG (FIG. 19) was characterized by NMR spectroscopy in the presence and absence of the tanshinone dHTSN1. As shown in FIG. 4A (black), the ¹⁵N-¹H HSQC spectrum of ¹⁵N-PscG in complex with PscE exhibited the typical feature of an α-helical protein where its resonance peaks distributed between 7.3 and 9.0 ppm in the proton dimension. The spectrum had a good dispersion except for the broadening of some resonance peaks in the center, indicating that parts of the PscG conformation were still flexible. Nevertheless, upon addition of dHTSN1 to the ¹⁵N-PscG-PscE complex at a molar ratio of 1:1:1, those flexible resonances became much shaper (gray), indicative of relatively strong interactions between dHTSN1 and the heterodimeric complex. Of note, these interactions also resulted in the broadening of a few other resonance peaks (marked in circles) beyond the center of the spectrum (FIG. 4A).

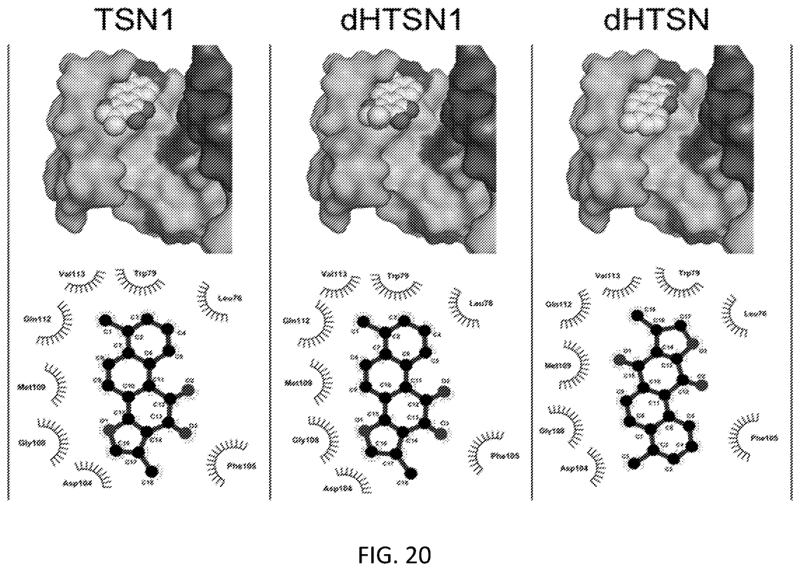

Significant changes to the side-chain amide resonance peaks of Trp residues were observed (the inset, FIG. 4A). There are four Trp residues in PscG, three of which, Trp 67, Trp73 and Trp79, are located on the same helix involved in PscF interactions (FIG. 4B). The four individual resonance peaks of Trp in the ¹⁵N-¹H HSQC spectrum were arbitrarily labelled as W1, W2, W3 and W4 (FIG. 4A, black). As shown in the inset of FIG. 4A (gray), upon binding of dHTSN1 to the PscG/PscE heterodimer, three amide resonance peaks of Trp became broadened and one remained unchanged. These results indicate that dHTSN1 binding is localized to the PscF-interacting helix of PscG, inducing direct and/or indirect changes in side-chain amide resonance to the three proximal Trp residues. In fact, molecular docking studies pinpointed Trp79, among other residues of PscG (FIG. 20), to be directly involved in tanshinone interactions (FIG. 4C).

### Example 6: Tanshinones Block the Secretion of the T3SS Effector ExoS In Vitro

The cytotoxicity of Pseudomonas aeruginosa against host immune cells and epithelial cells is dependent on its T3SS, through which four exotoxins (effector proteins) are unleashed into the host cytoplasm: ExoS, ExoT, ExoU and ExoY. ExoS and ExoT are homologous exotoxins with GTPase activating and ADP ribosyltransferase activities, capable of disrupting the actin cytoskeleton and inducing apoptotic cell death, while ExoU has phospholipase A₂ activity that induces rapid necrotic cell death via membrane lysis. It is anticipated that inhibitors of the biogenesis of the Pseudomonas aeruginosa T3 SS needle will shut down the exotoxin transport machinery, preventing or reducing the cytotoxic and pathogenic effects of Pseudomonas aeruginosa on host cells and tissues.

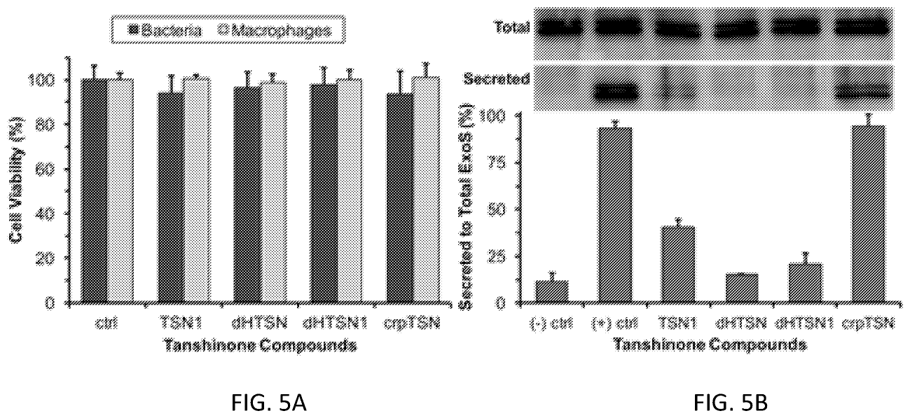

Infection of mouse macrophage cell line J774A.1 by the Pseudomonas aeruginosa reference strain PAO1 (ExoS, T, Y only) was used as an in vitro model to study the effects of tanshinones on Pseudomonas aeruginosa cytotoxicity and pathogenicity. None of the tanshinone compounds at 100 μM were directly bactericidal or bacteriostatic against PAO1, nor were they growth-inhibitory against macrophages (FIG. 5A). However, Western blot analysis showed that treatment with the three active tanshinone compounds at 100 μM each of PAO1, grown under low-calcium conditions to transcriptionally activate the T3SS, significantly reduced the secretion of ExoS (FIG. 5B). Consistent with the biochemical findings, dHTSN and dHTSN1 were more active than TSN1 in inhibiting ExoS secretion, while crpTSN showed no inhibitory activity. These data confirmed that an impaired biogenesis of the Pseudomonas aeruginosa T3SS needle could lead to a decrease in secretion of bacterial exotoxins and other virulence factors.

### Example 7: Tanshinones Reduce Cytotoxicity of Pseudomonas aeruginosa PAO1 to Macrophages and Inhibit Intracellular Bacterial Survival

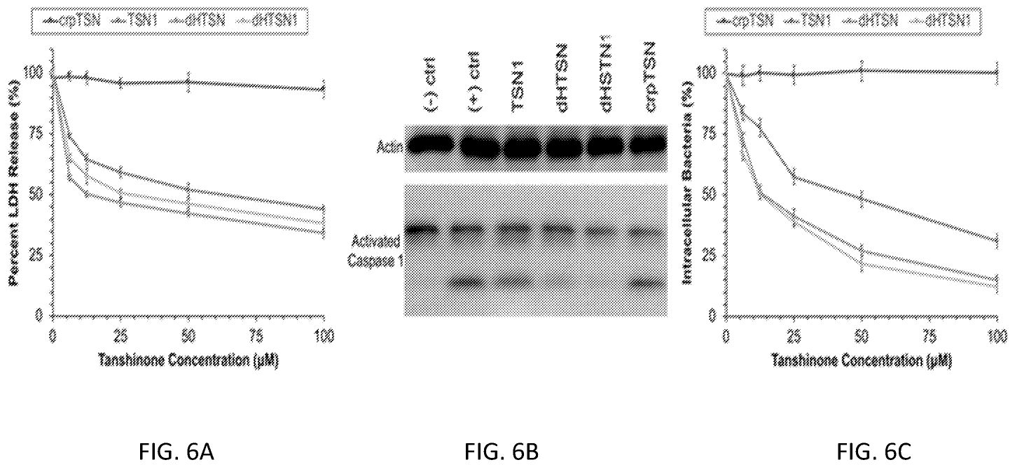

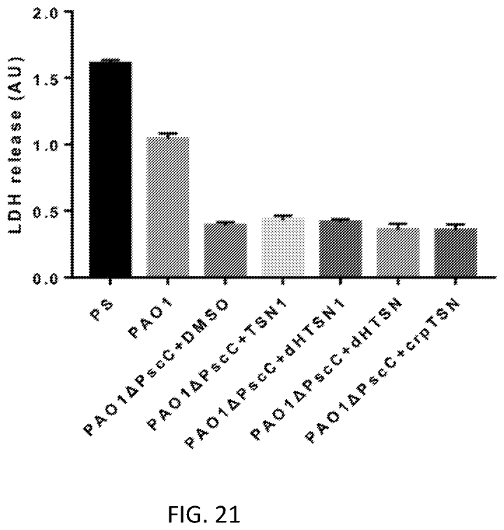

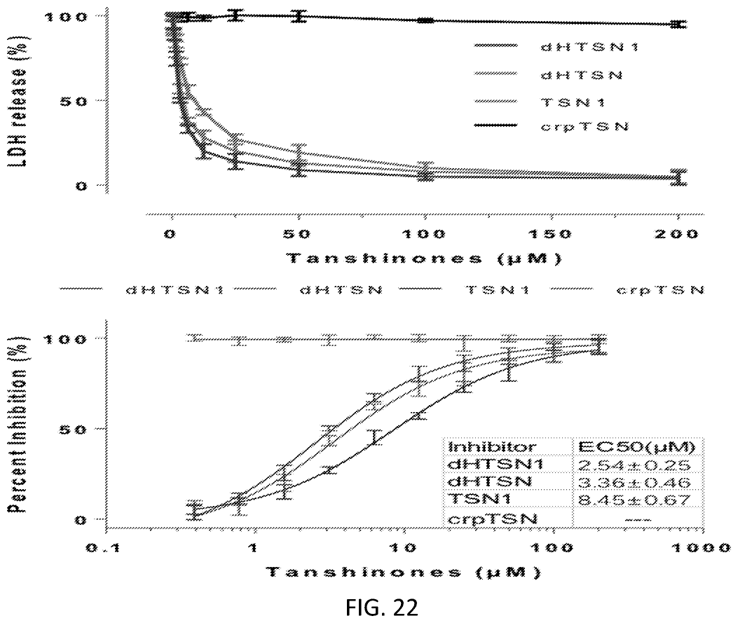

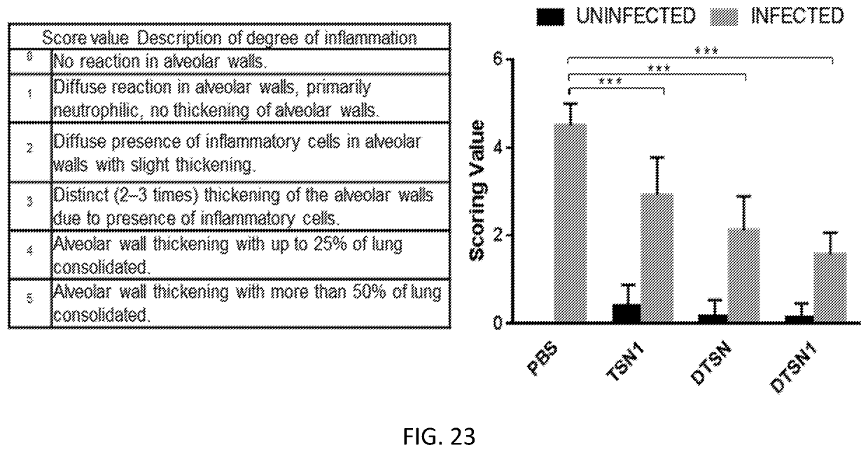

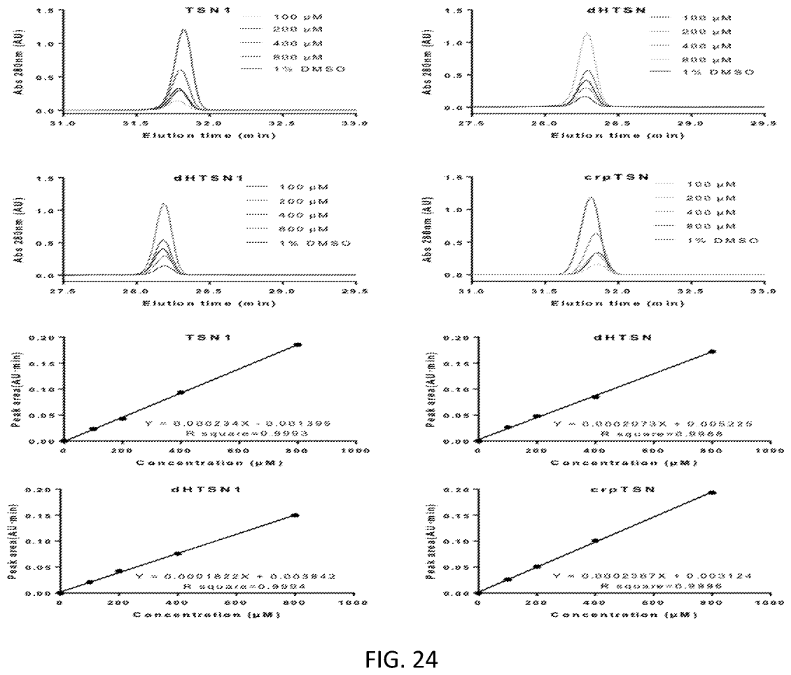

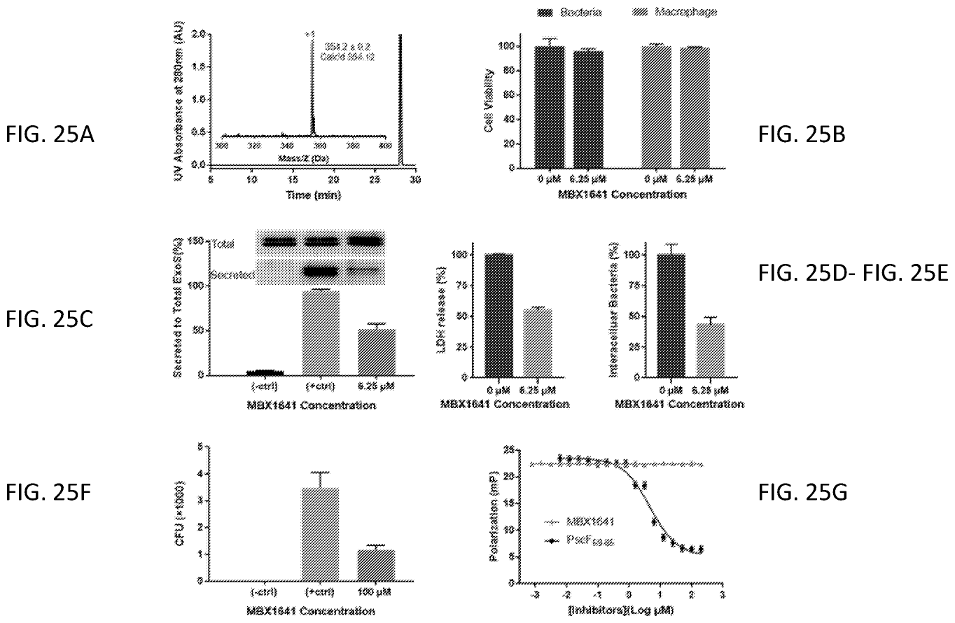

Macrophages infected by phagocytosed Pseudomonas aeruginosa undergo rapid cell death (pyroptosis) mediated by pro-inflammatory caspase-1, which is activated via Nod-like receptor signaling by bacterial flagellin injected into the cytosol by the T3SS. J774A.1 cells were infected with PAO1 in the presence of various tanshinone compounds and cytotoxicity was quantified by measuring the release into the medium of the cytoplasmic enzyme lactate dehydrogenase (LDH) by dying macrophages. As shown in FIG. 6A, TSN1, dHTSN and dHTSN1 inhibited Pseudomonas aeruginosa-induced cell lysis in a dose dependent manner, whereas crpTSN was inactive. Western blot analysis implied a reduction in activated caspase-1 as the plausible cause for the survival of infected macrophages (FIG. 6B), consistent with functional inhibition of the T3SS by tanshinones. Notably, less than 70% of inhibition of the cytotoxicity of PAO1 to macrophages was achieved by the two most active tanshinones dHTSN and dHTSN1 at 100 μM (EC₅₀=12.5 and 25 μM, respectively) (FIG. 6A), suggesting a significant residual cytotoxic effect (at the level of ˜30% LDH release) that could not be neutralized by tanshinone treatment. Since PAO1 and tanshinones were added simultaneously to macrophages before incubation, this persistent basal cytotoxicity was obviously independent of the future status of the biogenesis of the T3SS needle, and likely arose from pyroptosis of macrophages induced by phagocytosed bacteria harboring T3SS-independent cytotoxic factors. Results from an identical LDH assay using the mutant strain PAO1 ΔpscC constructed, which is defective in the T3SS due to the lack of the outer membrane ring protein PscC, confirmed that PAO1 ΔpscC also induced LDH release from macrophages, albeit at a reduced level compared with PAO1 (FIG. 21). In fact, tanshinone treatment had no effect on PAO1 ΔpscC-induced LDH release (FIG. 21), consistent with the fact that tanshinones act on the T3SS. Interestingly, when LDH released from dying macrophages treated with PAO1 was normalized against that with PAO1 ΔpscC, the EC₅₀ values of dHTSN and dHTSN1 were in the neighborhood of 3 μM (FIG. 22). Thus, the lack of an appropriate “negative” control strain in the LDH assay could artificially underestimate tanshinone activity.

Despite being a bona fide extracellular pathogen, phagocytosed Pseudomonas aeruginosa can transiently survive and even replicate in macrophages, a cellular event enabled by bacterial virulence factors that subvert antibacterial effector functions of macrophage. Recent imaging studies revealed that Pseudomonas aeruginosa PAO1, engulfed by macrophages into phagosomal vacuoles, can subsequently escape into the cytoplasm, where it ultimately induces cell lysis (Preeti Garai et al., bioRxiv 389718; doi: https://doi.org/10.1101/389718). Strikingly, both phagosomal escape and intracellular bacteria-mediated cell lysis are strongly dependent on ExoS—the very T3SS effector tanshinones block. PAO1-infected J774A.1 cells at 2 h post infection were treated with gentamicin to kill off extracellular bacteria, and quantified intracellular bacteria from lysed cells. As shown in FIG. 6C, the three active tanshinones dose-dependently inhibited intracellular bacterial survival, confirming that tanshinones can indeed protect macrophage function by inhibiting ExoS secretion to prevent phagosomal escape of and cell lysis mediated by phagocytosed PAO1.

It is worth noting that Pseudomonas aeruginosa can invade and actively multiply in epithelial cells in vitro and in vivo, where ExoS promotes its intracellular survival after invasion by (1) helping Pseudomonas aeruginosa avoid lysosomal degradation, and (2) creating membrane blebs as a replicative niche for the bacterium. Paradoxically, ExoS had long been thought to be capable of preventing Pseudomonas aeruginosa from being internalized or endocytosed by epithelial cells through destabilization of the actin cytoskeleton. More recent studies, however, have reconciled these contradictory findings by linking the purported anti-internalization activity of ExoS to the use of “artificial” reporter systems, including ectopic expression of ExoS without bacteria or in trans expression of ExoS in the background of PA103, an effector-null cytotoxic strain of Pseudomonas aeruginosa. When ExoS is natively encoded in Pseudomonas aeruginosa strains such as PAO1, it does not prevent bacterial internalization into epithelial cells.

### Example 8: In Vivo Efficacy of Tanshinones in a Murine Model of Acute Pneumonia

Phagocytic macrophages and neutrophils play critical roles in bacterial clearance during acute Pseudomonas aeruginosa infection in vivo. To subvert their antibacterial defense, Pseudomonas aeruginosa has evolved T3SS-dependent mechanisms to lyse macrophages and impair neutrophil function. Production of reactive oxygen species (ROS) by neutrophils is critical for intracellular killing of phagocytosed bacteria. A recent study demonstrated that the T3SS effectors ExoS and ExoT secreted by PAO1 independently inhibit ROS production in human neutrophils. Thus, inhibition of the biogenesis of the Pseudomonas aeruginosa T3SS needle should improve phagocytic functions of macrophages and neutrophils, leading to efficient bacterial clearance from infected host.

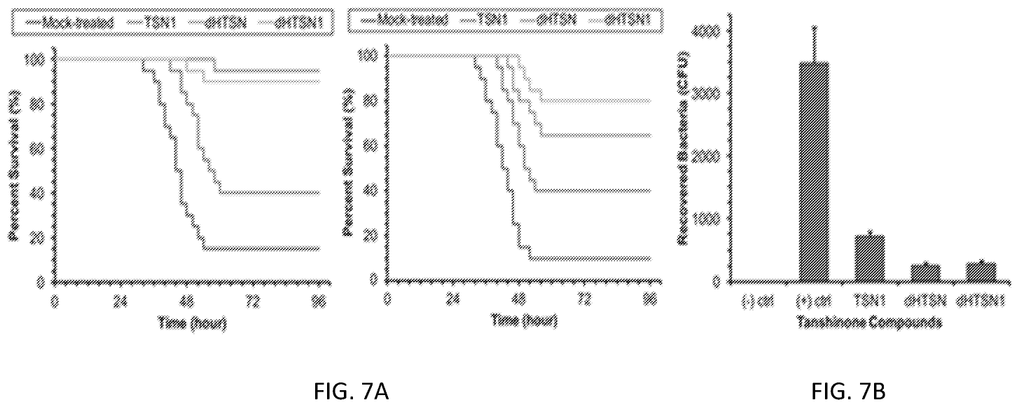

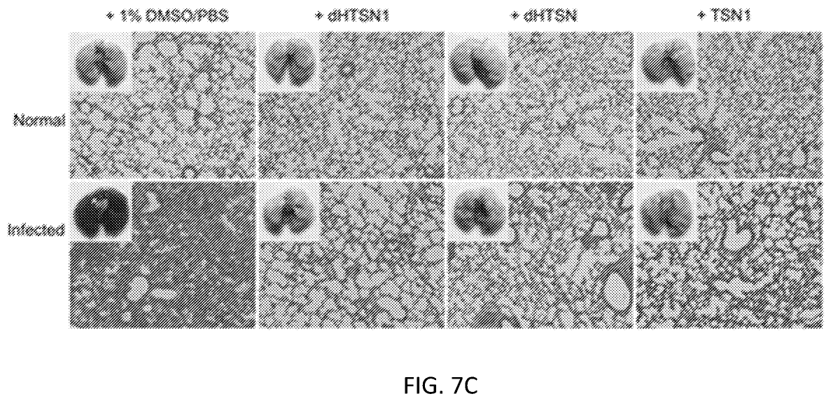

It was investigated whether or not tanshinones could protect against Pseudomonas aeruginosa infection in vivo using a murine model of acute pneumonia. As shown in FIG. 7A (left), without treatment, 70% of C57BL/6J mice intranasally inoculated with 1×10⁷ CFU of PAO1 died of acute lung infection within 48 h. While TSN1 at 100 μM given at the time of infection and every 12 h thereafter was significantly protective, dHTSN and dHTSN1 dramatically prolonged animal survival with over 90% of infected mice surviving beyond 96 h. To administer tanshinones in a clinically relevant setting, another in vivo efficacy study was performed using the same murine model of acute pneumonia where the first intranasal injection of tanshinones was made 8 h after the animals had been challenged with PAO1. As shown in FIG. 7A (right), while 85% of mice in the mock-treated group died of infection within 48 h, only 40%, 20% and 5% died in the groups treated with TSN, dHTSN and dHTSN1, respectively. Further, with dHTSN1 treatment, 80% of infected mice survived beyond 96 h.

In a separate in vivo experiment where infected mice received a single-dose treatment at the time of infection, PAO1 in the bronchoalveolar lavage sampled at 18 h post infection was quantified; active tanshinones significantly reduced bacterial burden in the lung (FIG. 7B) —an outcome likely arising, at least in part, from bacterial clearance by functional phagocytes afforded by an impaired Pseudomonas aeruginosa T3SS. Consistent with these findings, H&E staining of the lungs from mock-treated mice at 18 h post infection revealed extensive cellular infiltration and tissue damage that occluded the airways (FIGS. 7C, S16). By contrast, treatment by tanshinones and dHTSN1 in particular significantly reduced inflammation as evidenced by minimal infiltration of neutrophils into the alveolar spaces of the lungs (FIGS. 7C, S16). Taken together, our in vivo data support the premise that tanshinones prevent lung pathology associated with Pseudomonas infection by inhibiting the secretion via the T3SS of bacterial virulence factors.

### Example 9: Other Known Inhibitors of the T3SS

Of note, many studies have already validated the T3SS as an attractive drug target for antibiotic discovery and development. Two recent articles provide a comprehensive review of novel strategies for the treatment of Pseudomonas aeruginosa infections, including the targeting of the T3SS. Passive and active immunization with T3SS structural and effector proteins can prevent or reduce T3SS-induced bacterial virulence in vitro and in vivo. Although various cellular reporter assays coupled with library screening led to the identification of some small molecule inhibitors of the T3SS of relatively low potency, the lack of understanding of precise molecular targets and mechanisms of action has hampered their further development. Phenoxyacetamides are the only known class of compounds that block both the T3SS-mediated secretion and translocation of Pseudomonas aeruginosa effectors through binding to PscF to interfere with its multimerization. A prototypic phenoxyacetamide compound MBX-1641 in our in vitro and in vivo assays was tested and found functionally comparable to dHTSN and dHTSN1. As shown in FIG. S18, MBX-1641 inhibited the secretion of ExoS in PAO1, the cytotoxicity of PAO1 to murine macrophages, and the intracellular proliferation of PAO1 as well. Further, MBX-1641 reduced bacterial burden in the bronchoalveolar lavage of PAO1-infected mice (FIG. S18F). In contrast to tanshinones, however, MBX-1641 had no effect on the binding of PscF to PscE-PscG as analyzed by fluorescence polarization (FIG. S18G). These findings indicate that phenoxyacetamides and tanshinones mechanistically differ as inhibitors of the Pseudomonas aeruginosa T3 SS.

### CONCLUSIONS

Selective tanshinones have been determined as mechanistically defined inhibitors of the biogenesis of the Pseudomonas aeruginosa T3SS needle. Biochemical and biophysical as well as in vitro and in vivo functional studies of tanshinones validated these natural herbal compounds as promising drug candidates for the development of a novel class of antibiotics for the treatment of multidrug-resistant Pseudomonas aeruginosa infections. As an active ingredient in traditional Chinese medicine widely used to treat cardiovascular and cerebrovascular diseases with a demonstrable safety profile in humans, tanshionones may be used directly, upon conclusion of a comprehensive toxicology study to ascertain the safety of individual compounds, to alleviate Pseudomonas aeruginosa-associated pulmonary infections without inducing antibiotic resistance. Our work demonstrated the feasibility of targeting the biogenesis of the T3SS needle for antibiotic discovery by developing a sensitive fluorescence polarization assay for automated HTS of library compounds. Since the T3SS is highly conserved in many other pathogenic Gram-negative bacteria such as E. coli, Salmonella, Shigella, Yersinia, Vibrio, Burkholderia, and Chlamydia, our strategy for antibiotic discovery may have broad implications in combating antibiotic resistance.

The results described in Examples 1-9 were obtained using the following materials and methods.

Experimental Procedures

Materials. Boc-amino acids were purchased from Peptides Institute (Japan). Boc-Leu-OCH₂-PAM resin and p-methyl-BHA (MBHA) resin were purchased from Applied Biosystems (Foster City, Calif., USA). N,N-Dimethylformamide (DMF), Dichloromethane (DCM), N,N-Diisopropylethylamine (DIEA), Dimethyl sulfoxide (DMSO), methanol, 4-mercaptophenylacetic acid (MPAA), tris-(2-carboxyethyl) phosphine (TCEP), p-cresol and HPLC grade acetonitrile were purchased from Sigma-Aldrich (St. Louis, Mo., USA). Hydrogen fluoride (HF) was purchased from APK (Shanghai, China). Trifluoroacetic acid (TFA) was purchased from HaloCarbon (River Edge, N.J., USA). Tanshinone IIA, dihydrotanshinone 1, tanshinone 1, cryptotanshinone were purchased from Nature Standard (Shanghai, China). Ammonium glycyrrhizinate, astragaloside A, baicalein, curculigoside, ginsenoside Rb, ginsenoside Re, osthol, panaxadiol, quercetin, dihydrotanshinone were provided as generous gifts by Dr. Sha Liao of Northwestern University of China. All natural herbal compounds were prepared in DMSO (5-10 mM) and stored in dark at room temperature for no more than 1 month.

Peptide synthesis. All peptides were synthesized using the optimized HBTU activation/DIEA in situ neutralization protocol developed by Kent and colleagues for Boc-chemistry solid phase peptide synthesis (SPPS). Peptide cleavage from resin and side chain deprotection reactions were performed at 0-4° C. for 1 h in HF. After precipitation with cold ether, crude products were purified to homogeneity on a preparative HPLC using a C18 reversed-phase column. The molecular masses were ascertained by electrospray ionization mass spectrometry (ESI-MS).

Native chemical ligation. Native chemical ligation reactions were carried out in 0.1 M phosphate buffer containing 6 M guanidine hydrochloride (GuHCl), 100 mM MPAA and 40 mM TCEP, pH 7.4. Thz was used instead of Cys to protect the δ-mercaptolysine at position 26 of PscG to avoid an intramolecular head-to-tail ligation reaction. The Thz ring opened upon treatment of the peptide by MeONH₂.HCl at pH ˜4 for 12 h. The Trp(CHO) was deprotected using 20% piperidine and 20% tBUSH for 30 min.

Chemical synthesis of MBX1641. Compound MBX 1641 was prepared directly from 2-(2,4-dichlorophenoxy) propanoic acid and 3,4-methylenedioxybenzylamine (Alfa Aesar) as reported. Crude products were purified to homogeneity on a preparative HPLC using a C4 reversed-phase column. The molecular mass was ascertained by ESI-MS.

Heterotrimeric complex co-folding and characterization. Protein folding was achieved by dissolving the polypeptides (at the same molar ratio) in 6 M GuHCl at 1 mg/mL, followed by a 6-fold dilution with phosphate buffered saline (PBS) containing 0.5 mM TCEP, pH 7.4, and an overnight dialysis against the same buffer. After dialysis, the protein complex was analyzed by size exclusion chromatography on an ÄKTA protein purification system using a Superdex 75 column at a flow rate of 0.5 ml/minute at room temperature. The apparent molecular weights were calculated according to the standard calibration curve. The protein complex was also analyzed by reverse phase HPLC on a Waters XBridge C18 column (4.6×150 mm, 3.5 μm), and its molecular mass ascertained by ESI-MS.

Fluorescence polarization (FP) and FP-based competitive binding assays. All fluorescence polarization assays were done using black, low protein binding 96-well plates (Thermo Fisher Scientific) in a total volume of 100 μl per well of 10 mM Tris buffer containing 150 mM NaCl and 1 mM EDTA, pH 7.0, unless indicated otherwise. After a gentle mixing and incubation for 3 h, FP readings were taken at 470 nm (excitation) and 530 nm (emission) wavelengths on a Tecan Infinite M2000 fluorescence plate reader. Nonlinear regression analyses were performed to give rise to Kd and IC₅₀ values as previously described.

For direct binding of PscF to PscG or PscE-PscG, equal volumes of FAM-PscF⁶⁹⁻⁸⁵ (400 nM) and serially diluted PscG or PscE-PscG (0-64 μM) were mixed. For initial screening, 95 μl of FAM-PscF⁶⁹⁻⁸⁵-PscE-PscG (100 nM) in the assay buffer was mixed with 5 μl of small molecule inhibitor in DMSO to a final molar concentration of 1, 10, 100, or 1000 μM. 5% DMSO and 6 M GuHCl in the final assay buffer were used as negative and positive controls, respectively. For secondary screening or competitive binding assays, 95 μl of FAM-PscF⁶⁹⁻⁸⁵-PscE-PscG (100 nM) in the assay buffer was mixed with 5 μl of serially diluted PscF⁶⁹⁻⁸⁵ or various tanshinone analogs (0-200 μM).

Isothermal titration calorimetry (ITC). ITC was used to determine the binding affinity-K_{D}, enthalpy change-ΔH, and binding stoichiometry-n of the interaction between molecules. All ITC experiments were performed on a MicroCal ITC 200 at 25° C. in 10 mM Tris, 150 mM NaCl, 1 mM EDTA, pH 7.0. The concentration of PscF⁵⁴⁻⁸⁵ and PscE were 300 μM each, and the concentration of PscG and PscE-PscG 30 μM each. From these initial measurements, Gibbs energy changes ΔG and entropy changes ΔS can be determined using the formula: ΔG=−RT InKα=ΔH−TΔS.

Circular dichroism (CD) spectroscopy and thermal denaturation. CD spectra of proteins at a concentration of 200 nM in 10 mM phosphate buffer (pH 7.4) were obtained at room temperature on a circular dichroism spectrometer (Jasco, Easton, Md.) using a 1-mm quartz cuvette. Protein thermal denaturation was carried out in PBS using a JASCO circular dichroism spectrometer equipped with temperature controller. 2.5 mL of protein solution (10 μM) prepared in PBS, pH 7.4, was aliquoted into a 3 mL cuvette. Under constant stirring, measurement was taken at one-degree interval between 25° C. and 90° C., at a heating rate of 1° C. per minute. After each 1-minute heating, the solution in the cuvette was waited for 20 s before signals were detected over a 16 s period. Heating and data acquisition were fully automated with the control software provided by JASCO. Data processing was performed as previously described.

Expression of ¹⁵N-labelled PscG and NMR Characterization of ¹⁵N-labelled PscG in complex with PscE and dHTSN1. PscG was uniformly labeled with ¹⁵N MOPS medium containing 1 g/L ¹⁵NH₄Cl as the sole source and BME vitamins (Sigma, USA). Cells were harvested by centrifugation at 6000 g for 30 min, resuspended in a lysis buffer containing 6 M urea, and subjected to a 1-min sonication, followed by two-cycle homogenization at 4° C. The lysates were centrifuged at 20,000 rpm for 30 min, and the supernatant was loaded onto a 10 ml Ni-NTA agarose column (Qiagen, USA). The elute was concentrated to 10-12 ml under denaturation conditions, followed by purification on a Sep-Pak C18 column. Peak fractions containing ¹⁵N-PscG were lyophilized. The ¹⁵N-PscG protein samples were dissolved in a buffer containing 20 mM sodium phosphate (pH 7.4), 10 mM NaCl, and mixed with PscE or PscE and dHTSN1 at an equal molar ratio. The mixtures were dialyzed against the same buffer overnight and then concentrated to ˜300 μl.

All NMR samples were prepared in an NMR buffer containing 20 mM sodium phosphate (pH 7.4), 100 mM NaCl, 0.1% NaN₃, 10% D₂O and 2 mM DTT. The final protein concentrations were approximately 26 μM and 47.8 μM for ¹⁵N-PscG-PscE and ¹⁵N-PscG-PscE-dHTSN1 complexes, respectively. All NMR spectra were collected at 25° C. on a Bruker Avance 700 MHz spectrometer equipped with a triple-resonance pulse-field gradient probe. ¹⁵N-¹H HSQC NMR spectra were recorded in the echo-antiecho mode for quadrature detection. All datasets were acquired with 2048 complex points in t₂ and 256 complex points in t₁. Data were processed using Topspin software and displayed using NMRViewJ software.

Molecular docking. The 3-dimensional structure of the heterotrimeric complex of PscE-PscF-PscG was used for molecular docking. PscF was removed from the heterotrimeric complex to prepare the molecular target for docking, so were the four N-terminal residues (GSHM) in PscE that showed high values of b-factor in the heterotrimeric complex. The three active tanshinone compounds and the PscE-PscG complex were prepared for docking using the AutoDockTools software suite. The AutoGrid module was used to create a grid box with center at 44.174, 28.238, 18.306 and size of 106, 102, 82 points along the XYZ directions with a spacing of 0.375 Å. Docking calculations were performed with AutoDock4.2.6 using the Lamarkian Genetic algorithm with a population size of 150, and the number of evaluations and generations set to 10 000 000. 100 docking runs were performed for each compound and docked conformations were clustered using AutoDockTools with a cut-off of 2.0 Å, yielding the largest cluster exceeded 95% of the total runs.

Bacterial strain and cell line and growth conditions. Pseudomonas aeruginosa isolate PAO1 and its mutant strain PAO1 ΔpscC were cultured in Luria broth (LB) at 37° C. The mouse macrophage cell line J774A.1 (ATCC TIB-67) was cultured in Dulbecco's Modified Eagle Medium (DMEM) supplemented with 10% fetal bovine serum (FBS) and incubated at 37° C. in 5% CO₂.

Bactericidal activity assay. PAO1 overnight cultures were diluted 1:100 in LB, in the presence of tanshinone inhibitors (100 μM compound, 2% DMSO). After a 3 h-incubation with mild agitation, bacteria were diluted and plated. Bactericidal activity was determined by colony counting and normalized against the activity under mock treatment (2% DMSO only). Results are represented as the mean±SD percentage of input bacteria of three independent experiments.

Cytotoxicity of tanshinones to macrophages. A total of 1×10⁴ J774A.1 cells were seeded into each well of a 96-well plate and grown for 24 hours. Different tanshinone inhibitors (100 μM compound, 2% DMSO) were added and incubated at 37° C. in 5% CO₂ for 8 hours followed by CCK-8 cell viability assay (Beyotime, C0038) according to the manufacturer instructions. The control group were treated with 2% DMSO. Optical density (OD) was measured at 450 nm. Percentage of cell viability was calculated as following:

Cell viability=(OD_{treated cells}−OD_{blank})/(OD_{control cells}−OD_{blank})×100%. Average results of three independent experiments are shown as mean±SD.

Inhibition of T3SS-mediated effector secretion. For T3SS induction, PAO1 overnight cultures were diluted to an OD at 600 nm of 0.3 in LB containing 5 mM EGTA and 20 mM MgCl₂, in the presence of tanshinone inhibitors (100 μM compound, 2% DMSO), followed by an incubation with mild agitation of additional 3-4 hours until the cultures reached an OD value of 1.5. NC group were cultured in LB and 2% DMSO, PC group were cultured in LB containing 5 mM EGTA, 20 mM MgCl₂ and 2% DMSO. Culture supernatant was collected by centrifugation at 4000 g for 15 min at 4° C. and secreted proteins were concentrated by adding ice-cold trichloroacetic acid to a final concentration of 10% (V/V). Following a 2 hours' incubation on ice, the pellets were washed twice with cold acetone and suspended in an SDS-PAGE sample buffer (with 2-mercaptoethanol) according to the BCA protein assay protocol. Secreted and total proteins (supernatant and pelleted bacteria) were analyzed by immunoblotting with an anti-ExoS antibody (Agrisera, AS05056, at 1:4000 dilution) and corresponding HRP-conjugated secondary antibody. The blots were semi-quantified using ImageJ 1.51 k (from http://imagej.nih.gov/ij). The results were expressed as the mean±SD percentage of secreted ExoS out of total ExoS of three independent experiments.

Cytoplasmic lactate dehydrogenase (LDH) release assay. 1×10⁴ J774a cells were seeded into each well of a 96-well plate and grown for 24 h before infection. One hour before the infection, cell culture medium was changed into serum-free medium and PAO-1 from mid-exponential phase was added to the cells at a multiplicity of infection (MOI) of 8. In the presence of different concentration of tanshinone inhibitors (0-100 M compound, 2% DMSO), bacteria/cells mixtures were incubated for 5 hours. LDH released into supernatant was detected by LDH detection kit (Beyotime, C0017) as instructed by the manufacturer. Results were normalized against the LDH released by PAO-1-infected cells with mock treatment (2% DMSO). Average results of three independent experiments are shown as mean±SD.

Caspase 1-mediated pyroptosis of PAO1-infected macrophages. 2×10⁵ J774a cells were seeded into each well of a 6-well plate and grown for 24 h before infection. One hour before the infection, cell culture medium was changed into serum-free medium and PAO-1 from mid-exponential phase was added to the cells at a multiplicity of infection (MOI) of 8. In the presence (100 μM compound, 2% DMSO) or absence (2% DMSO) of tanshinone inhibitors, bacteria/cells mixtures were incubated for 3 hours at 37° C. in 5% CO₂. Cells were collected with lysis buffer (with 2-mercaptoethanol) according to the BCA protein quantification protocol (Beyotime, P0017), and subjected to 15% SDS-PAGE gel and immunoblotting with anti-pro Caspase 1+p10+p12 antibody (Abcam, ab179515, at 1:1000 dilution) and corresponding HRP-conjugated secondary antibody. Beta-actin was used as internal control.

Quantification of bacterial internalization. 5×10⁴ J774a cells were seeded into each well of a 24-well plate and grown for 24 h before infection. One hour before the infection, cell culture medium was changed into serum-free medium and PAO-1 from mid-exponential phase was added to the cells at a multiplicity of infection (MOI) of 8. In the presence of different concentration of tanshinone inhibitors (0-100 μM compound, 2% DMSO), bacteria/cells mixtures were incubated for 2 hours and washed, then treated with gentamicin-containing (50 μg/ml) medium for another 2 hours before being lysed for plating. Internalized bacteria were defined as the total number of intracellular bacteria in cells (extracellular bacteria were killed by gentamicin, a cell-impermeable antibiotic). Results were normalized against the intracellular bacteria number by PAO-1-infected cells with mock treatment (2% DMSO). Average results of three independent experiments are shown as mean±SD.

Animal studies. 6-week-old female C57BL/6J mice used in this study were acquired from the Experimental Animal Center of Xian Jiaotong University. All the animals were maintained in animal care facilities in the School of Life Science and Technology, and provided with food and water ad libitum. The animal studies were approved by the Committee on Animal Research and Ethics, Xi'an Jiaotong University.

C57BL/6J mice were lightly anesthetized with inhaled sevoflurane and infected by intranasal instillation of PAO1 (1×10⁷ CFU in 20 μL PBS) after lightly anesthetized with inhaled sevoflurane. Tanshinone inhibitors were administrated to the animals along with bacterial inoculation (100 μM, 1% DMSO). PBS containing 1% DMSO was used as mock treatment. 18 hours after infection, animals were sacrificed and bronchoalveolar lavages were collected and plated to obtain the bacterial counts in the lavages. The lungs of sacrificed mice were then isolated and fixed in 10% buffered formalin, paraffin embedded and hematoxylin-eosin-stained for histopathological examination. Pathological scores of the tissues were assigned according to the degree of inflammation.

For survival study, tanshinone inhibitors (100 μM, 1% DMSO, in 10 μL PBS) were administrated to the infected animals intranasally at the time of infection, or 8 h after infection, and every 12 hours till the death of the animal or the end of the experiment. PBS containing 1% DMSO was used as mock treatment.

Statistical Analysis. The data were collected from at least three independent experiments in triplicate or quadruplicate, unless otherwise indicated. Data were combined and represented as mean±SEM or mean±SD as indicated. Results were analyzed by various statistical tests using GraphPad Prism version 7. p<0.05 was considered statistically significant. Microscopy images are representative of at least two independent experiments.

Supporting Information

Chemical synthesis and chromatographic and mass spectrometric characterization of PscE, PscF and PscG; size exclusion chromatography of PscE-PscF-PscG; thermal denaturation of PscE, PscF, PscG, PscE-PscG and PscE-PscF-PscG; quantification of PscG interaction with PscF or PscE by isothermal titration calorimetry and/or fluorescence polarization; fluorescence polarization-based initial screening of natural herbal compounds; effects of tanshinones on the conformation and stability of PscE-PscF-PscG; tanshinone derivatives and their fluorescence spectrometric properties; SDS-PAGE analysis of recombinant PscG expressed in E. coli; molecular docking of tanshinones in PscE-PscG; cytotoxicity of PAO1 and PAO1 ΔpscC; solubility of tanshinones in 1% DMSO; functional and mechanistic characterization of MBX1641

### REFERENCES

1. O'Neill, J. Review on antimicobial resistance. Antimicrobial reisistance: tackling a crisis for the health and wealth of nations; The UK Government and the Wellcome Trust: 2014.
2. Blair, J. M.; Webber, M. A.; Baylay, A. J.; Ogbolu, D. O.; Piddock, L. J., Molecular mechanisms of antibiotic resistance. Nat Rev Microbiol 2015, 13 (1), 42-51.3.
3. Tenover, F. C., Mechanisms of antimicrobial resistance in bacteria. Am J Med 2006, 119 (6 Suppl 1), S3-10; discussion S62-70.
4. Allen, R. C.; Popat, R.; Diggle, S. P.; Brown, S. P., Targeting virulence: can we make evolution-proof drugs? Nat Rev Microbiol 2014, 12 (4), 300-308.
5. Dickey, S. W.; Cheung, G. Y. C.; Otto, M., Different drugs for bad bugs: antivirulence strategies in the age of antibiotic resistance. Nat Rev Drug Discov 2017, 16 (7), 457-471.
6. Silva, L. N.; Zimmer, K. R.; Macedo, A. J.; Trentin, D. S., Plant natural products targeting bacterial virulence factors. Chem Rev 2016, 116 (16), 9162-9236.
7. Coburn, B.; Sekirov, I.; Finlay, B. B., Type III secretion systems and disease. Clin Microbiol Rev 2007, 20 (4), 535-549.
8. Hueck, C. J., Type III protein secretion systems in bacterial pathogens of animals and plants. Microbiol Mol Biol Rev 1998, 62 (2), 379-433.
9. Hauser, A. R., The type III secretion system of Pseudomonas aeruginosa: infection by injection. Nat Rev Microbiol 2009, 7 (9), 654-665.
10. Sawa, T.; Yahr, T. L.; Ohara, M.; Kurahashi, K.; Gropper, M. A.; Wiener-Kronish, J. P.; Frank, D. W., Active and passive immunization with the Pseudomonas V antigen protects against type III intoxication and lung injury. Nat Med 1999, 5 (4), 392-398.
11. Hauser, A. R.; Cobb, E.; Bodi, M.; Mariscal, D.; Valles, J.; Engel, J. N.; Rello, J., Type III protein secretion is associated with poor clinical outcomes in patients with ventilator-associated pneumonia caused by Pseudomonas aeruginosa. Crit Care Med 2002, 30 (3), 521-528.
12. Roy-Burman, A.; Savel, R. H.; Racine, S.; Swanson, B. L.; Revadigar, N. S.; Fujimoto, J.; Sawa, T.; Frank, D. W.; Wiener-Kronish, J. P., Type III protein secretion is associated with death in lower respiratory and systemic Pseudomonas aeruginosa infections. J Infect Dis 2001, 183 (12), 1767-1774.
13. Smith, R. S.; Wolfgang, M. C.; Lory, S., An adenylate cyclase-controlled signaling network regulates Pseudomonas aeruginosa virulence in a mouse model of acute pneumonia. Infect Immun 2004, 72 (3), 1677-1684.
14. Wagner, S.; Sommer, R.; Hinsberger, S.; Lu, C.; Hartmann, R. W.; Empting, M.; Titz, A., Novel strategies for the treatment of Pseudomonas aeruginosa infections. J Med Chem 2016, 59 (13), 5929-5969.
15. Anantharajah, A.; Mingeot-Leclercq, M. P.; Van Bambeke, F., Targeting the type three secretion system in Pseudomonas aeruginosa. Trends Pharmacol Sci 2016, 37 (9), 734-749.
16. Abrusci, P.; McDowell, M. A.; Lea, S. M.; Johnson, S., Building a secreting nanomachine: a structural overview of the T3SS. Curr Opin Struct Biol 2014, 25, 111-117.
17. Quinaud, M.; Chabert, J.; Faudry, E.; Neumann, E.; Lemaire, D.; Pastor, A.; Elsen, S.; Dessen, A.; Attree, I., The PscE-PscF-PscG complex controls type III secretion needle biogenesis in Pseudomonas aeruginosa. J Biol Chem 2005, 280 (43), 36293-36300.
18. Quinaud, M.; Ple, S.; Job, V.; Contreras-Martel, C.; Simorre, J. P.; Attree, I.; Dessen, A., Structure of the heterotrimeric complex that regulates type III secretion needle formation. Proc Natl Acad Sci USA 2007, 104 (19), 7803-7808.
19. Dawson, P. E.; Kent, S. B., Synthesis of native proteins by chemical ligation. Annu Rev Biochem 2000, 69, 923-960.
20. Dawson, P. E.; Muir, T. W.; Clark-Lewis, I.; Kent, S. B., Synthesis of proteins by native chemical ligation. Science 1994, 266 (5186), 776-779.
21. Smith, D. S.; Eremin, S. A., Fluorescence polarization immunoassays and related methods for simple, high-throughput screening of small molecules. Anal Bioanal Chem 2008, 391 (5), 1499-1507.
22. Huang, X.; Aulabaugh, A., Application of fluorescence polarization in HTS assays. Methods Mol Biol 2009, 565, 127-143.
23. Owicki, J. C., Fluorescence polarization and anisotropy in high throughput screening: perspectives and primer. J Biomol Screen 2000, 5 (5), 297-306.
24. Stover, C. K.; Pham, X. Q.; Erwin, A. L.; Mizoguchi, S. D.; Warrener, P.; Hickey, M. J.; Brinkman, F. S.; Hufnagle, W. O.; Kowalik, D. J.; Lagrou, M.; Garber, R. L.; Goltry, L.; Tolentino, E.; Westbrock-Wadman, S.; Yuan, Y.; Brody, L. L.; Coulter, S. N.; Folger, K. R.; Kas, A.; Larbig, K.; Lim, R.; Smith, K.; Spencer, D.; Wong, G. K.; Wu, Z.; Paulsen, I. T.; Reizer, J.; Saier, M. H.; Hancock, R. E.; Lory, S.; Olson, M. V., Complete genome sequence of Pseudomonas aeruginosa PAO1, an opportunistic pathogen. Nature 2000, 406 (6799), 959-964.
25. Vallis, A. J.; Yahr, T. L.; Barbieri, J. T.; Frank, D. W., Regulation of ExoS production and secretion by Pseudomonas aeruginosa in response to tissue culture conditions. Infect Immun 1999, 67 (2), 914-920.
26. Miao, E. A.; Ernst, R. K.; Dors, M.; Mao, D. P.; Aderem, A., Pseudomonas aeruginosa activates caspase 1 through Ipaf. Proc Natl Acad Sci USA 2008, 105 (7), 2562-2567.
27. Li, K.; Xu, C.; Jin, Y.; Sun, Z.; Liu, C.; Shi, J.; Chen, G.; Chen, R.; Jin, S.; Wu, W., SuhB is a regulator of multiple virulence genes and essential for pathogenesis of Pseudomonas aeruginosa. MBio 2013, 4 (6), e00419-00413.
28. Price, J. V.; Vance, R. E., The macrophage paradox. Immunity 2014, 41 (5), 685-693.
29. Fleiszig, S. M.; Zaidi, T. S.; Fletcher, E. L.; Preston, M. J.; Pier, G. B., Pseudomonas aeruginosa invades corneal epithelial cells during experimental infection. Infect Immun 1994, 62 (8), 3485-3493.
30. Fleiszig, S. M.; Zaidi, T. S.; Pier, G. B., Pseudomonas aeruginosa invasion of and multiplication within corneal epithelial cells in vitro. Infect Immun 1995, 63 (10), 4072-4077.
31. Angus, A. A.; Evans, D. J.; Barbieri, J. T.; Fleiszig, S. M., The ADP-ribosylation domain of Pseudomonas aeruginosa ExoS is required for membrane bleb niche formation and bacterial survival within epithelial cells. Infect Immun 2010, 78 (11), 4500-4510.
32. Angus, A. A.; Lee, A. A.; Augustin, D. K.; Lee, E. J.; Evans, D. J.; Fleiszig, S. M., Pseudomonas aeruginosa induces membrane blebs in epithelial cells, which are utilized as a niche for intracellular replication and motility. Infect Immun 2008, 76 (5), 1992-2001.
33. Heimer, S. R.; Evans, D. J.; Stern, M. E.; Barbieri, J. T.; Yahr, T.; Fleiszig, S. M., Pseudomonas aeruginosa utilizes the type III secreted toxin ExoS to avoid acidified compartments within epithelial cells. PLoS One 2013, 8 (9), e73111.
34. Kroken, A. R.; Chen, C. K.; Evans, D. J.; Yahr, T. L.; Fleiszig, S. M. J., The impact of ExoS on Pseudomonas aeruginosa internalization by epithelial cells is independent of fleQ and correlates with bistability of type three secretion system gene expression. MBio 2018, 9 (3), 1-21.
35. Koh, A. Y.; Priebe, G. P.; Ray, C.; Van Rooijen, N.; Pier, G. B., Inescapable need for neutrophils as mediators of cellular innate immunity to acute Pseudomonas aeruginosa pneumonia. Infect Immun 2009, 77 (12), 5300-5310.
36. Kurahashi, K.; Sawa, T.; Ota, M.; Kajikawa, O.; Hong, K.; Martin, T. R.; Wiener-Kronish, J. P., Depletion of phagocytes in the reticuloendothelial system causes increased inflammation and mortality in rabbits with Pseudomonas aeruginosa pneumonia. Am J Physiol Lung Cell Mol Physiol 2009, 296 (2), L198-209.
37. Vareechon, C.; Zmina, S. E.; Karmakar, M.; Pearlman, E.; Rietsch, A., Pseudomonas aeruginosa effector ExoS inhibits ROS production in human neutrophils. Cell Host Microbe 2017, 21 (5), 611-618 e615.
38. Baer, M.; Sawa, T.; Flynn, P.; Luehrsen, K.; Martinez, D.; Wiener-Kronish, J. P.; Yarranton, G.; Bebbington, C., An engineered human antibody fab fragment specific for Pseudomonas aeruginosa PcrV antigen has potent antibacterial activity. Infect Immun 2009, 77 (3), 1083-1090.
39. Milla, C. E.; Chmiel, J. F.; Accurso, F. J.; VanDevanter, D. R.; Konstan, M. W.; Yarranton, G.; Geller, D. E.; Group, K. B. S., Anti-PcrV antibody in cystic fibrosis: a novel approach targeting Pseudomonas aeruginosa airway infection. Pediatr Pulmonol 2014, 49 (7), 650-658.
40. Gu, L.; Zhou, S.; Zhu, L.; Liang, C.; Chen, X., Small-molecule inhibitors of the type III secretion system. Molecules 2015, 20 (9), 17659-17674.
41. Aiello, D.; Williams, J. D.; Majgier-Baranowska, H.; Patel, I.; Peet, N. P.; Huang, J.; Lory, S.; Bowlin, T. L.; Moir, D. T., Discovery and characterization of inhibitors of Pseudomonas aeruginosa type III secretion. Antimicrob Agents Chemother 2010, 54 (5), 1988-1999.
42. Berube, B. J.; Murphy, K. R.; Torhan, M. C.; Bowlin, N. O.; Williams, J. D.; Bowlin, T. L.; Moir, D. T.; Hauser, A. R., Impact of type III secretion effectors and of phenoxyacetamide inhibitors of type III secretion on abscess formation in a mouse model of Pseudomonas aeruginosa infection. Antimicrob Agents Chemother 2017, 61 (11), 1-15.
43. Bowlin, N. O.; Williams, J. D.; Knoten, C. A.; Torhan, M. C.; Tashjian, T. F.; Li, B.; Aiello, D.; Mecsas, J.; Hauser, A. R.; Peet, N. P.; Bowlin, T. L.; Moir, D. T., Mutations in the Pseudomonas aeruginosa needle protein gene pscF confer resistance to phenoxyacetamide inhibitors of the type III secretion system. Antimicrob Agents Chemother 2014, 58 (4), 2211-2220.
44. Zhou, L.; Zuo, Z.; Chow, M. S., Danshen: an overview of its chemistry, pharmacology, pharmacokinetics, and clinical use. J Clin Pharmacol 2005, 45 (12), 1345-1359.
45. Schnolzer, M.; Alewood, P.; Jones, A.; Alewood, D.; Kent, S. B., In situ neutralization in Boc-chemistry solid phase peptide synthesis. Rapid, high yield assembly of difficult sequences. Int J Pept Protein Res 1992, 40 (3-4), 180-193.
46. Zhan, C.; Varney, K.; Yuan, W.; Zhao, L.; Lu, W., Interrogation of MDM2 phosphorylation in p53 activation using native chemical ligation: the functional role of Ser17 phosphorylation in MDM2 reexamined. Journal of the American Chemical Society 2012, 134 (15), 6855-6864.
47. Zhan, C.; Zhao, L.; Wei, X.; Wu, X.; Chen, X.; Yuan, W.; Lu, W. Y.; Pazgier, M.; Lu, W., An ultrahigh affinity d-peptide antagonist of MDM2. J Med Chem 2012, 55 (13), 6237-6241.
48. Lu, W. Y.; Starovasnik, M. A.; Dwyer, J. J.; Kossiakoff, A. A.; Kent, S. B.; Lu, W., Deciphering the role of the electrostatic interactions involving Gly70 in eglin C by total chemical protein synthesis. Biochemistry 2000, 39 (13), 3575-3584.
49. Dolcetti, R.; Giagulli, C.; He, W.; Selleri, M.; Caccuri, F.; Eyzaguirre, L. M.; Mazzuca, P.; Corbellini, S.; Campilongo, F.; Marsico, S.; Giombini, E.; Muraro, E.; Rozera, G.; De Paoli, P.; Carbone, A.; Capobianchi, M. R.; Ippolito, G.; Fiorentini, S.; Blattner, W. A.; Lu, W.; Gallo, R. C.; Caruso, A., Role of HIV-1 matrix protein p17 variants in lymphoma pathogenesis. Proc NatlAcad Sci USA 2015, 112 (46), 14331-14336.
50. He, W.; Mazzuca, P.; Yuan, W.; Varney, K.; Bugatti, A.; Cagnotto, A.; Giagulli, C.; Rusnati, M.; Marsico, S.; Diomede, L.; Salmona, M.; Caruso, A.; Lu, W.; Caccuri, F., Identification of amino acid residues critical for the B cell growth-promoting activity of HIV-1 matrix protein p17 variants. Biochim Biophys Acta Gen Subj 2018, 1863 (1), 13-24.
51. Morris, G. M.; Huey, R.; Lindstrom, W.; Sanner, M. F.; Belew, R. K.; Goodsell, D. S.; Olson, A. J., AutoDock4 and AutoDockTools4: Automated docking with selective receptor flexibility. J Comput Chem 2009, 30 (16), 2785-2791.
52. Szarka, R. J.; Wang, N.; Gordon, L.; Nation, P. N.; Smith, R. H., A murine model of pulmonary damage induced by lipopolysaccharide via intranasal instillation. J Immunol Methods 1997, 202 (1), 49-57.
53. Shi, J. R.; Mao, L. G.; Jiang, R. A.; Qian, Y.; Tang, H. F.; Chen, J. Q., Monoammonium glycyrrhizinate inhibited the inflammation of LPS-induced acute lung injury in mice. Int Immunopharmacol 2010, 10 (10), 1235-1241.
54. Huang, P.; Lu, X.; Yuan, B.; Liu, T.; Dai, L.; Liu, Y.; Yin, H., Astragaloside IV alleviates E. coli-caused peritonitis via upregulation of neutrophil influx to the site of infection. Int Immunopharmacol 2016, 39, 377-382.
55. Zeng, Z.; Qian, L.; Cao, L.; Tan, H.; Huang, Y.; Xue, X.; Shen, Y.; Zhou, S., Virtual screening for novel quorum sensing inhibitors to eradicate biofilm formation of Pseudomonas aeruginosa. Appl Microbiol Biotechnol 2008, 79 (1), 119-126.
56. Nie, Y.; Dong, X.; He, Y.; Yuan, T.; Han, T.; Rahman, K.; Qin, L.; Zhang, Q., Medicinal plants of genus Curculigo: traditional uses and a phytochemical and ethnopharmacological review. J Ethnopharmacol 2013, 147 (3), 547-563.
57. Yu, S.; Zhou, X.; Li, F.; Xu, C.; Zheng, F.; Li, J.; Zhao, H.; Dai, Y.; Liu, S.; Feng, Y., Microbial transformation of ginsenoside Rb1, Re and Rg1 and its contribution to the improved anti-inflammatory activity of ginseng. Sci Rep 2017, 7 (1), 138.
58. Rosselli, S.; Maggio, A.; Bellone, G.; Formisano, C.; Basile, A.; Cicala, C.; Alfieri, A.; Mascolo, N.; Bruno, M., Antibacterial and anticoagulant activities of coumarins isolated from the flowers of Magydaris tomentosa Planta Med 2007, 73 (2), 116-120.
59. Liu, Y. H.; Liu, Y. F.; Guo, X. X., Current studies on anti-endotoxic chemical components of traditional Chinese medicine in China. Acta Pharmacol Sin 2001, 22 (12), 1071-1077.
60. Woznicka, E.; Kuzniar, A.; Nowak, D.; Nykiel, E.; Kopacz, M.; Gruszecka, J.; Golec, K., Comparative study on the antibacterial activity of some flavonoids and their sulfonic derivatives. Acta Pol Pharm 2013, 70 (3), 567-571.
61. Wang, X.; Morris-Natschke, S. L.; Lee, K. H., New developments in the chemistry and biology of the bioactive constituents of Tanshen. Med Res Rev 2007, 27 (1), 133-148

A number of patent and non-patent publications are cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

1. A method of treating or preventing a Gram-negative bacterial infection in a subject in need thereof, the method comprising administering to the subject a therapeutically effective amount of an inhibitor of Type 3 Secretion System (T3SS), wherein the inhibitor of T3SS is selected from the group consisting of a tanshinone, tanshinone analog, and the pharmaceutically acceptable salts, solvates, hydrates, cocrystals, or prodrugs thereof.

2. The method of claim 1, wherein the tanshinone is tanshinone 1 (TSN1).

3. The method of claim 1, wherein the tanshinone analog is dihydrotanshinone 1 (dHTSN1) or dihydrotanshinone (dHTSN).

4. The method of any one of claims 1-3, wherein the Gram-negative bacteria is Pseudomonas, Escherichia, Salmonella, Shigella, Yersinia, Vibrio, Burkholderia, or Chlamydia.

5. The method of any one of claims 1-4, wherein the Gram-negative bacteria is Escherichia coli or Pseudomonas aeruginosa.

6. The method of any one of claims 1-5, wherein the Gram-negative bacteria is Pseudomonas aeruginosa.

7. The method of any one of claims 1-6, wherein the bacterial infection is a lung infection, skin infection, soft tissue infection, gastrointestinal infection, urinary tract infection, meningitis, or sepsis.

8. The method of any one of claims 1-7, wherein the bacterial infection is pneumonia.

9. The method of any one of claims 1-8, wherein the subject is a human.

10. A pharmaceutical composition for the treatment or prevention of a Gram-negative bacterial infection in a subject in need thereof, the composition comprising an inhibitor of Type 3 Secretion System (T3SS), wherein the inhibitor of T3SS is selected from the group consisting of a tanshinone, tanshinone analog, and the pharmaceutically acceptable salts, solvates, hydrates, cocrystals, or prodrugs thereof.

11. The pharmaceutical composition of claim 10, wherein the composition further comprises a pharmaceutically acceptable carrier.

12. The pharmaceutical composition of any one of claims 10-11, wherein the tanshinone is tanshinone 1 (TSN1).

13. The pharmaceutical composition of any one of claims 10-11, wherein the tanshinone analog is dihydrotanshinone 1 (dHTSN1) or dihydrotanshinone (dHTSN).

14. The pharmaceutical composition of any one of claims 10-13, wherein the Gram-negative bacteria is Pseudomonas, Escherichia, Salmonella, Shigella, Yersinia, Vibrio, Burkholderia, or Chlamydia.

15. The pharmaceutical composition of claim 14, wherein the Gram-negative bacteria is Escherichia coli or Pseudomonas aeruginosa.

16. The pharmaceutical composition of claim 15, wherein the Gram-negative bacteria is Pseudomonas aeruginosa

17. The pharmaceutical composition of any one of claims 10-16, wherein the bacterial infection is a lung infection, skin infection, soft tissue infection, gastrointestinal infection, urinary tract infection, meningitis, or sepsis.

18. The pharmaceutical composition of any one of claims 10-17, wherein the bacterial infection is pneumonia.

19. The pharmaceutical composition of any one of claims 10-18, wherein the subject is a human.

20. A method of inhibiting treating or preventing a Gram-negative bacterial infection in a subject in need thereof, the method comprising administering to the subject a therapeutically effective amount of an inhibitor of Type 3 Secretion System (T3SS), wherein the inhibitor of T3SS blocks interaction between a T3SS needle protein and a T3SS chaperone protein.

21. The method of claim 20, wherein the T3SS needle protein is PscF and the T3SS chaperone protein is PscE-PscG.

22. The method of claim 21, wherein the inhibitor of T3SS competes for binding to PscE-PscG with PscF.

23. The method of claim 22, wherein the inhibitor of T3SS competes for binding with an IC50 between 0.5 μM to 3 μM.

24. The method of any one of claims 22-23, wherein the inhibitor of T3SS binds one or more residues on PscG selected from Trp79, Trp 67, Trp73, and Trp31.

25. The method of any one of claims 22-24, wherein the inhibitor of T3SS binds residue Trp79 on PscG.

26. The method of any one of claims 21-25, wherein the inhibitor of T3SS is a tanshinone or tanshinone analog.

27. The method of any one of claims 21-26, wherein the bacterial infection is pneumonia.

28. The method of any one of claims 21-27, wherein the subject is human.

29. A pharmaceutical composition for the treatment or prevention of a Gram-negative bacterial infection in a subject in need thereof, the composition comprising an inhibitor of Type 3 Secretion System (T3SS), and a pharmaceutically acceptable carrier, wherein the inhibitor of T3SS blocks interaction between a T3SS needle protein and a T3SS chaperone protein.

30. The pharmaceutical composition of claim 29, wherein the T3SS needle protein is PscF and the T3SS chaperone protein is PscE-PscG.

31. The pharmaceutical composition of claim 30, wherein the inhibitor of T3SS competes for binding to PscE-PscG with PscF.

32. The pharmaceutical composition of claim 31, wherein the inhibitor of T3SS competes for binding with an IC50 between 0.5 μM to 3 μM.

33. The pharmaceutical composition of any one of claims 30-32, wherein the inhibitor of T3SS binds one or more residues on PscG selected from Trp79, Trp 67, Trp73, and Trp31.

34. The pharmaceutical composition of any one of claims 30-33, wherein the inhibitor of T3SS binds residue Trp79 on PscG.

35. The pharmaceutical composition of any one of claims 30-34, wherein the inhibitor of T3SS is a tanshinone or tanshinone analog.

36. The pharmaceutical composition of any one of claims 30-35, wherein the bacterial infection is a pneumonia.

37. The pharmaceutical composition of any one of claims 30-36, wherein the subject is human.

38. A method of identifying an inhibitor of Type 3 Secretion System (T3SS), the method comprising:
(a) adding a candidate agent to a composition comprising a protein complex in the T3SS, wherein a component of the protein complex is fluorescently labeled;
(b) determining the fluorescence polarization (FP) of the fluorescently labeled component;
wherein the candidate agent is identified as an inhibitor of T3SS if the FP is decreased relative to a reference FP level.

39. The method of claim 38, wherein the protein complex comprises a T3SS needle protein and a T3SS chaperone protein.

40. The method of claim 38 or 39, wherein the protein complex is PscF-PscE-PscG.

41. The method of claim 40, wherein the PscF is labeled with a fluorescent label.

42. The method of claim 41, wherein the PscF comprises the amino acid sequence set forth in SEQ ID NO: 1.

43. The method of any one of claims 38-42, wherein the reference FP level is the FP of the fluorescently labeled component without a candidate agent added to the composition.

44. The method of any one of claims 38-43, wherein the candidate agent is a small molecule compound.

45. The method of any one of claims 38-44, wherein the candidate agent is a tanshinone or tanshinone analog.

46. The method of any one of claims 38-45, wherein the method is high-throughput.

47. A method of treating or preventing bacterial infection in a subject in need thereof, the method comprising administering to the subject a therapeutically effective amount of an agent identified as an inhibitor of Type 3 Secretion System (T3SS) according to the method of any one of claims 38-46.

48. A pharmaceutical composition for the treatment or prevention of a bacterial infection in a subject in need thereof, the composition comprising an agent identified as an inhibitor of Type 3 Secretion System (T3SS) according to the method of any one of claims 38-46, and a pharmaceutically acceptable carrier.

49. A method of treating a Gram-negative bacterial infection in a subject in need thereof, the method comprising administering to the subject a therapeutically effective amount of an inhibitor of Type 3 Secretion System (T3SS) selected from the group consisting of tanshinone 1 (TSN1), dihydrotanshinone 1 (dHTSN1), dihydrotanshinone (dHTSN), and the pharmaceutically acceptable salts, solvates, hydrates, cocrystals, or prodrugs thereof.

50. A pharmaceutical composition for the treatment of a Gram-negative bacterial infection in a subject in need thereof, the composition comprising an inhibitor of Type 3 Secretion System (T3SS) selected from the group consisting of tanshinone 1 (TSN1), dihydrotanshinone 1 (dHTSN1), dihydrotanshinone (dHTSN), and the pharmaceutically acceptable salts, solvates, hydrates, cocrystals, or prodrugs thereof, and a pharmaceutically acceptable carrier.
